(19) United States
(12) Patent Application Publication
(10) Pub. No.: US 2021/0403586 A1
(43) Pub. Date: Dec. 30, 2021

(54) MUTANT OF RANKL AND PHARMACEUTICAL COMPOSITION COMPRISING SAME FOR PREVENTING OR TREATING OSTEOPOROSIS

(71) Applicant: INDUSTRY-ACADEMIC COOPERATION FOUNDATION CHOSUN UNIVERSITY, Dong-gu (KR)

(72) Inventors: Won Bong LIM, Buk-gu (KR); Young Jong Ko, Seo-g (KR); Min Eon Park, Seo-gu (KR); Bo Ra Kim, Jangseong-gun (KR); Gwang Chul Lee, Gwangsan-gu (KR)

(21) Appl. No.: 17/317,252

(22) Filed: May 11, 2021

Related U.S. Application Data
(63) Continuation-in-part of application No. PCT/KR2019/009480, filed on Jul. 30, 2019.

(30) Foreign Application Priority Data
Nov. 12, 2018 (KR) 10-2018-0138305

(51) Int. Cl. C07K 16/28; C12N 15/63; A61P 19/08

(52) U.S. Cl. CPC C07K 16/2875; A61K 2039/505; A61P 19/08; C12N 15/63

(57) ABSTRACT

Provided are a mutant of RANKL protein that acts in vivo as an antigen to induce production of an anti-RANKL antibody so as to inhibit production of osteoclasts, but does not induce differentiation of osteoclasts while binding to RANK, and a composition for preventing or treating a metabolic bone disease, the composition comprising the mutant of RANKL protein as an active ingredient.

### TECHNICAL FIELD

The present disclosure relates to a mutant of RANKL, and a pharmaceutical composition for preventing or treating osteoporosis, the pharmaceutical composition including the mutant of RANKL.

### BACKGROUND ART

Osteoporosis refers to a condition that weakens bones, making them more likely to break, in which bone mineral density (BMD) is 2.5 or less, or T-score (the number of standard deviations from the mean BMD of healthy adults) is −2.5 or less due to decreased bone mass and quality. When the bond density is excessively decreased, fractures can easily occur even with a small impact. Osteoporosis is not the symptom itself but is known to lead to unhealthy lives by limiting physical activities for a long time due to various fractures caused by bone weakness, particularly, femoral fractures or spinal fractures, and as a result, responsible for 15% of elderly deaths.

Human bones consist of osteoblasts, osteocytes, and osteoclasts. Among them, osteoblasts play a role in forming bone tissues through a proliferation stage, a bone matrix maturation stage, and a mineralization stage. In addition, osteoclasts play a role in bone resorption. In adult bones after growth, a bone remodeling process occurs while bone resorption and formation, in which old bones are removed by osteoclasts and new bones are replaced by osteoblasts, are continuously regenerated. For example, osteoblasts regulate differentiation of osteoclasts responsible for bone resorption through secretion of substances, such as receptor activator of nuclear factor-kappa B ligand (RANKL) and its decoy receptor osteoprotegerin (OPG), and thus homeostasis of bone metabolism is maintained in the body. When the homeostasis of bone metabolism is broken by a specific cause, metabolic bone diseases occur such as osteoporosis, osteodystrophy, bone fractures, etc.

As described above, osteoclasts are responsible for bone resorption in bone-related disorders, and anti-cytokine antibodies such as denosumab are known to be effective in treating osteoporosis. However, the high cost of manufacturing and immunogenicity caused by multiple antibody doses remain a major problem in such anti-cytokine immunotherapy. To overcome the problems of such immunotherapy resistance and therapy using anti-cytokine antibody, the present disclosure proposes an immunotherapy for applying a mutant of RANKL as an immunogen to osteoporosis to induce anti-cytokine antibodies.

### DESCRIPTION OF EMBODIMENTS

### Technical Problem

An aspect provides a mutant of receptor activator of nuclear factor-kappa B ligand (RANKL) protein.

Another aspect provides an antibody produced by the mutant of RANKL protein.

Still another aspect provides a nucleic acid molecule encoding the mutant of RANKL protein.

Still another aspect provides a vector comprising the nucleic acid molecule encoding the mutant of RANKL protein.

Still another aspect provides a host cell comprising the vector.

Still another aspect provides a pharmaceutical composition for preventing or treating a metabolic bone disease, the pharmaceutical composition comprising the mutant of RANKL protein as an active ingredient.

Still another aspect provides use of the mutant of RANKL protein in preparing the pharmaceutical composition for preventing or treating a metabolic bone disease.

Still another aspect provides a method of preventing or treating a metabolic bone disease, the method comprising administering the mutant of RANKL protein to a subject.

### Solution to Problem

An aspect provides a mutant of receptor activator of nuclear factor-kappa B ligand (RANKL) protein.

The term ‘RANKL protein’ may refer to receptor activator of nuclear factor-kappa B (NF-κB) ligand (RANKL). RANKL is known as a type II membrane protein and is a member of the tumor necrosis factor (TNF) super family. RANKL is an apoptosis regulator gene, and is able to control cell proliferation by modifying protein levels of Id4, Id2 and cyclin D1. RANKL may be expressed in several tissues and organs, including skeletal muscle, thymus, liver, colon, small intestine, adrenal arteries, osteoblasts, mammary epithelial cells, prostate, and pancreas.

NF-κB is a group of proteins involved in inflammatory response regulation, immune system regulation, apoptosis, cell proliferation, and differentiation of epithelial cells. NF-κB regulates expression of various genes and plays a pivotal role in the intracellular signaling pathways. When RANKL binds to the receptor activator of nuclear factor kappa-B (NF-κB) (RANK), RANK is activated, which may lead to activation of NF-κB, mitogen-activated protein kinase (MAPK), activating protein 1 (AP-1), and nuclear factor of activated T cells (NFATc1).

The ‘mutant’ may refer to an organism or a protein that has undergone mutation. The ‘mutation’ may mean that a DNA molecule in which genetic information is recorded is different from the original. When a mutation occurs, a change occurs in a protein produced by the gene, which may lead to a change in the genetic trait.

The types of mutations may be classified according to their size and how they function. Mutations may include, as mutations occurring at a nucleotide level, ‘point mutation’ resulting in conversion of one nucleotide, ‘insertion mutation’ resulting in insertion of some nucleotides into the original nucleotide sequence, and ‘deletion mutation’ resulting in loss of some original nucleotides, and as mutations occurring at a chromosome level, ‘gene duplication’, ‘gene deletion’, ‘chromosome inversion’, ‘interstitial deletion’, ‘chromosome translocation’, and ‘loss of heterozygosity’.

The RANKL protein may be a mutant in which one or more amino acids in an amino acid sequence are substituted.

Another aspect provides a mutant having one or more substitutions selected from the group consisting of substitution of arginine (Arg) for lysine (Lys) at position 180, substitution of any one of isoleucine (Ile), leucine (Leu), and asparagine (Asn) for aspartic acid (Asp) at position 189, substitution of lysine (Lys) for arginine (Arg) at position 190, substitution of phenylalanine (Phe) or tyrosine (Try) for histidine (His) at position 223, and substitution of phenylalanine (Phe) or tyrosine (Try) for histidine (His) at position 224 at the N-terminus of RANKL protein comprising an amino acid sequence of SEQ ID NO: 1.

Still another aspect provides a mutant having one or more substitutions selected from the group consisting of substitution of arginine (Arg) for lysine (Lys) at position 181, substitution of any one of isoleucine (Ile), leucine (Leu), and asparagine (Asn) for aspartic acid (Asp) at position 190, substitution of lysine (Lys) for arginine (Arg) at position 191, substitution of phenylalanine (Phe) or tyrosine (Try) for histidine (His) at position 224, and substitution of phenylalanine (Phe) or tyrosine (Try) for histidine (His) at position 225 at the N-terminus of RANKL protein comprising an amino acid sequence of SEQ ID NO: 2.

SEQ ID NO: 1 is an amino acid sequence of RANKL protein (mRANKL) of mouse (Mus musculus). The amino acid substitution site of the RANKL protein may be related to a site that binds to the receptor of RANK.

SEQ ID NO: 2 is an amino acid sequence of RANKL protein (hRANKL) of human (Homo sapiens). The amino acid sequence of human RANKL protein is 317 in total length, and contains one additional amino acid than the amino acid sequence of a mouse with a length of 316, and the RANKL substitution site may be located one position downstream of the amino acid sequence of mouse. The amino acids at the substitution sites may be identical to each other.

The mutant may be administered to a subject to produce an antibody.

Therefore, the present disclosure further provides an antibody produced by the mutant of RANKL protein.

Antibody (immunoglobulin) may be a substance that causes an antigen-antibody reaction by specific binding to an antigen. The antibody may be a polyclonal antibody, a monoclonal antibody, a minibody, a domain antibody, a bispecific antibody, an antibody mimic, a chimeric antibody, an antibody conjugate, a human antibody or a humanized antibody, or any fragment thereof.

After administered to a subject, the mutant may induce formation of an anti-RANKL antibody in the subject. In other words, the mutant may be an antigen which is a substance acting as an immunogen for active immunity. The produced antibody may undergo an antigen-antibody reaction with RANKL in a subject. When the concentration of RANKL in the subject is decreased by the antibody, the binding of RANKL and RANK is reduced, thereby inhibiting differentiation of osteoclasts.

Another aspect provides a nucleic acid molecule encoding the mutant of RANKL protein.

In one specific embodiment, the mutant of RANKL may have a point mutation in the nucleic acid encoding the amino acids of RANKL protein. ‘Point mutation’ is a mutation that occurs at a nucleotide level, whereby one nucleotide is converted to prevent or modify production of a specific protein at the DNA transcription stage. Point mutation may exert the same effects as ‘silent mutation’ where the altered codon directs formation of the same amino acid as the existing codon, ‘missense mutation’ where the altered codon directs formation of different amino acids, and ‘nonsense mutation’ where amino acid formation is stopped or omitted due to the altered codon.

The nucleic acid molecule encoding the mutant of RANKL may comprise a nucleotide sequence of SEQ ID NO: 3 or 4.

The nucleotide sequence of SEQ ID NO: 3 may have one or more substitutions selected from the group consisting of substitution of CG for AA which are nucleotides at positions 676 to 677, substitution of ATCAAG for GATCGA which are nucleotides at positions 674 to 679, and substitution of TTT for CAC which are nucleotides at positions 803, 804, and 806 in a nucleotide sequence of a nucleic acid encoding the existing mouse RANKL.

The nucleotide sequence of SEQ ID NO: 4 may have one or more substitutions selected from the group consisting of substitution of CG for AA which are nucleotides at positions 669 to 670, substitution of ATCAA for GATCG which are nucleotides at positions 696 to 671, and substitution of TTT for CAC which are nucleotides at positions 798, 799, and 801 in a nucleotide sequence of a nucleic acid encoding the existing human RANKL.

In a specific embodiment, the point mutation of the nucleic acid encoding the RANKL protein may be induced by megaprimers. The megaprimer method is a kind of PCR method, which is used to complete a mutation by performing PCR in two stages, when a site to be mutated is located in the middle of the protein and thus it is difficult to complete the mutation at once. Specifically, when PCR amplification is induced by using a forward primer containing a nucleotide to be induced and a reverse primer containing a nucleotide of the C-terminal of a protein, DNA without information on the N-terminus upstream the forward primer may be obtained. In the next stage, when PCR is performed using a megaprimer as a reverse primer and oligo DNA containing the N-terminus of the protein as a forward primer, DNA encoding a full-length protein in which the mutation is induced at the desired position may be obtained.

The mutant may be in the form of a recombinant protein. The term ‘recombinant protein’ refers to a protein obtained by artificially expressing a ‘recombinant DNA’ in cells, which is a new DNA prepared by inserting a specific gene into a vector using a genetic recombination method. The ‘genetic recombination’ method refers to a technology that binds a DNA fragment of an arbitrary organism to another DNA molecule. Genetically, in most cases, genetic recombination may be accomplished through transformation, transduction, conjugation (crossing), and cell fusion.

Still another aspect provides a vector comprising the nucleic acid molecule encoding the mutant of RANKL. The vector is a DNA molecule used as an artificial vehicle of a nucleotide sequence. Its intracellular replication is possible, and gene expression may occur. In genetic engineering, a specific site of a vector may be digested using a restriction enzyme, and then a nucleotide sequence in need may be inserted thereto, which is then inserted into host cells, followed by culturing. The vector may serve as a vehicle into which a nucleotide sequence may be inserted. The nucleotide sequence may be exogenous or heterologous. Types of vectors include plasmids, cosmids, and viruses such as bacteriophages.

Still another aspect provides a host cell comprising the vector. The host cell includes eukaryotes and prokaryotes, and it refers to any transformable organism capable of replicating the vector or expressing a gene encoded by the vector. The host cell may be transfected or transformed by the vector, which means a process whereby an exogenous nucleic acid molecule is transferred or introduced into the host cell. The host cell may be represented by Escherichia coli (E. coli).

Still another aspect provides a pharmaceutical composition for diagnosing or treating a metabolic bone disease, the pharmaceutical composition comprising, as an active ingredient, the mutant of RANKL protein, or the antibody produced by the mutant.

The mutant of RANKL protein may have a substitution in the amino acid sequence of RANKL protein represented by SEQ ID NO: 1 or SEQ ID NO: 2. The site to be substituted is the same as the site described above.

The metabolic bone disease may be one or more selected from the group consisting of osteoporosis, osteodystrophy, and bone fracture. The osteoporosis may be caused by inducing differentiation of osteoclasts due to binding of RANKL to RANK.

Osteoclasts are cells that break down bones, and bone may be broken down by osteoclasts when the body needs to remove calcium from the bone. Osteoclasts may break down bones when there is a lack of calcium in the blood and the calcium in the bones needs to be supplied to the blood, or when finely fractured or cracked bones, or old bones need to be replaced with new bones. This imbalance between osteoclasts and bone-producing osteoblasts may lead to bone metabolic diseases such as osteoporosis.

The term “differentiation” refers to a phenomenon in which structures or functions are specialized to each other while cells divide and proliferate, i.e., cells, tissues, etc. of an organism change their shape or function in order to perform a task given to them.

The differentiation and activation of osteoclasts may be regulated by RANKL. Osteoclasts are formed as multinuclear bone resorptive osteoclasts when activation of RANK by RANKL in osteoclast progenitor cells stimulates TNF receptor-associated factors and sequentially activates NF-κB, mitogen-activated protein kinase (MAPK), activating protein 1 (AP-1), and nuclear factor of activated T cells 1 (NFATc1).

The mutant may be an antagonist of RANK receptors in a subject. An antagonist is a molecule that inhibits action of an agonist by binding to an active site of a receptor. Antagonists may be classified into receptor antagonists and non-receptor antagonists. Receptor antagonists bind to the active or allosteric site of a receptor, thereby inhibiting binding of an agonist to the receptor. In contrast, non-receptor antagonists have the ability to suppress a reaction induced by an agonist.

In addition, receptor antagonists include competitive antagonists that inhibit the action of agonists, and non-competitive antagonists that inhibit the action of agonists by affecting the number of receptors or irreversibly binding to receptors.

In one specific embodiment, the antagonist may include a competitive antagonist. After competitive antagonists reversibly bind to the active site of a receptor, they do not stabilize the structural changes required for receptor activation, unlike agonists. Accordingly, the receptor remains inactive and the binding to the agonist is blocked.

The mutant is a competitive antagonist and, like RANKL, is able to bind to RANK, but may not induce osteoclast differentiation even after binding. The mutant acts as an immunogen, but may not cause any physiological activity even when it binds to RANK. Therefore, as differentiation of osteoclasts is not induced, the composition may exhibit effects of preventing or treating a metabolic bone disease caused by RANKL expression.

Still another aspect provides a method of treating or preventing a metabolic bone disease, the method comprising administering, to a subject, a therapeutically effective amount of the pharmaceutical composition.

The subject may include humans.

The metabolic bone disease may be one or more selected from the group consisting of osteoporosis, osteodystrophy, and bone fracture, as described above. The osteoporosis may be caused by inducing production of osteoclasts due to binding of RANKL to RANK, as described above.

The dosage (effective amount) of the pharmaceutical composition according to one specific embodiment may be 0.01 mg to 10,000 mg, 0.1 mg to 1000 mg, 1 mg to 100 mg, 0.01 mg to 1000 mg, 0.01 mg to 100 mg, 0.01 mg to 10 mg, or 0.01 mg to 1 mg. However, the dosage may be variously prescribed depending on factors such as a formulation method, mode of administration, a patient's age, weight, sex, pathological conditions, diet, time of administration, route of administration, rate of excretion, and response sensitivity. Taking into account these factors, the dosage may be appropriately adjusted by those skilled in the art. Administration frequency may be once, or twice or more within the range of clinically acceptable side effects, and the site of administration may be one, two or more sites. For animals other than humans, a dosage that is the same as that of per kg in a human, or a dosage that is determined by, for example, conversion based on the volume ratio (e.g., average value) of organs (e.g., heart, etc.) of a target animal and a human, may be administered. Possible routes of administration may include oral, sublingual, parenteral (e.g., subcutaneous, intramuscular, intra-arterial, intraperitoneal, intrathecal, or intravenous), rectal, topical (including transdermal), inhalation, injection, or insertion of implantable devices or materials. As a target animal for the therapy according to one specific embodiment, a human and a mammal of interest may be exemplified, and specifically, it may include a human, a monkey, a mouse, a rat, a rabbit, sheep, a cow, a dog, a horse, a pig, etc.

The pharmaceutical composition according to one specific embodiment may include a pharmaceutically acceptable carrier and/or additive. The pharmaceutical composition may include, for example, sterile water, physiological saline, common buffers (phosphoric acid, citric acid, other organic acids, etc.), stabilizers, salts, antioxidants (ascorbic acid, etc.), surfactants, suspending agents, isotonic agents, preservatives, etc. For topical administration, the pharmaceutical composition may include a combination with organic compounds such as biopolymers, etc., and inorganic compounds such as hydroxyapatite, etc., specifically, collagen matrix, a polylactic acid polymer or copolymer, a polyethyleneglycol polymer or copolymer and chemical derivatives thereof, etc. When the pharmaceutical composition according to one specific embodiment is formulated into a dosage form suitable for injection, the mutant of RANKL or the antibody produced by the mutant is dissolved in a pharmaceutically acceptable carrier or frozen as a solution.

The pharmaceutical composition according to one specific embodiment may appropriately include suspensions, dissolution aids, stabilizers, isotonic agents, preservatives, anti-adhesion agents, surfactants, diluents, excipients, pH adjusting agents, pain relieving agents, buffers, reducing agents, anti-oxidants, etc., depending on its administration method or dosage form as needed. Pharmaceutically acceptable carriers and preparations suitable for the present disclosure, including those mentioned above, are described in detail in [Remington's Pharmaceutical Sciences, 19th ed., 1995]. The pharmaceutical composition according to one specific embodiment may be formulated by using pharmaceutically acceptable carriers and/or excipients according to methods which may be easily carried out by those skilled in the art, and thus the composition may be manufactured as a unit dosage form or incorporated into a multiple dose container. In this regard, the dosage forms may be in the form of a solution, suspension, or emulsion in oil or aqueous medium, or powders, granules, tablets, or capsules.

### Advantageous Effects of Disclosure

A mutant of RANKL according to one aspect may act as an immunogen producing an anti-RANKL antibody and as a competitive antagonist for RANK receptor, thereby effectively preventing or treating a metabolic bone disease.

### BRIEF DESCRIPTION OF DRAWINGS

FIG. 1 shows a full-length target region of 158 amino acids from 158 to 316 residues of mouse RANKL.

FIG. 2A shows amino acid sequence of the RANKL protein in human, mouse, and mutant RANKL transformants.

FIG. 2B shows SDS-PAGE of RANKL produced in E. coli after IPTG induction.

FIG. 2C shows effects of RANKL variants on the generation of tartrate-resistant acid phosphatase (TRAP)-positive multinucleated cells. TRAP-positive cells were visualized under a light microscope (100× magnification). The scale bar indicates 20 μm.

FIG. 2D shows quantification of TRAP-positive osteoclasts.

FIG. 2E shows effect of RANKL-MT treatment on the development of TRAP-positive multinucleated cells in the presence of RANKL-WT (75 ng/mL).

FIG. 2F shows counted osteoclasts (TRAP-positive cells). *P<0.05 and **P<0.01 when comparing RANKL-MT3 with others, respectively.

FIG. 2G shows effect of dose-dependent RANKL-MT3 treatment on bone resorption activity compared to RANKL-WT (75 ng/mL).

In FIG. 2H, resorption pits were quantified to investigate osteoclast activity. (i) TRAP, NFATc1, and OSCAR mRNA expression were analyzed by RT-PCR. The data were normalized to GAPDH expression and are shown as the mean ratio±SD from three separate experiments. *P<0.05 and ***P<0.001 when comparing RANKL-WT with RANKL-MT3.

FIG. 2I shows actin filaments formation of rhodamine—conjugated phalloidin—stained cells were visualized under a fluorescence microscope (200× magnification). The scale bar indicates 100 μm.

In FIG. 2J, TRAP, NFATc1, and OSCAR mRNA expression were analyzed by RT-PCR. The data were normalized to GAPDH expression and are shown as the mean ratio ±SD from three separate experiments. *P<0.05 and ***P<0.001 when comparing RANKL-WT with RANKL-MT3.

FIG. 3 shows insertion of cloned genes between Ndel and Xhol restriction enzyme sites in the multiple cloning site of pET30a vector.

FIG. 4A shows SDS-PAGE from the stepwise purification of RANKL produced in E. coli under IPTG induction (1(step): before induction, 2: induction, 3: supernatant, 4: cell debris, 5: column flow, 6: wash, 7: 2^{nd} wash, 8: 1^{st} elution, 9: 2^{nd} elution, 10: 3^{rd} elution).

FIG. 4B shows that wtRANKL injection increased the number of TRAP-positive multinucleated cells, and mtRANKL injection led to a reduction of TRAP-positive cells, wherein TRAP-positive cells were imaged under a light microscope (100× magnification) and the scale bar indicates 20 μm.

FIG. 4C shows osteoclasts (TRAP-positive cells) counted in the serum of wtRANKL-injected mice and mtRANKL-injected mice (p<0.05).

FIG. 5A shows a Micro-CT three-dimensional images of trabecular bone architectures of volume of interest (VOI) in tibias of PBS-treated mice, wtRANKL-treated mice, and mtRANKL-treated mice, wherein scanning for the proximal tibia was initiated proximally at the level of growth plate and the resolution was 19 μm in all three spatial dimensions.

FIG. 5B shows bone mineral density (BMD), bone volume, % bone volume, and trabecular thickness in PBS-treated mice, wtRANKL-treated mice, and mtRANKL-treated mice (p<0.05).

FIG. 6A shows results of Western blot analysis of mouse serum after treatment of non-immunized mice (left) and mtRANKL-immunized mice (right) with wtRANKL or mtRANKL.

FIG. 6B shows results of ELISA of serum levels of RANKL in PBS-injected mice, wtRANKL-injected mice, and mtRANKL-immunized mice with wtRANKL injection.

FIG. 7A shows a Micro-CT three-dimensional images of trabecular bone architectures of volume of interest (VOI) in tibias of PBS-injected mice, wtRANKL-injected mice, and mtRANKL-immunized mice with wtRANKL injection.

FIG. 7B shows bone mineral density, bone volume, % bone volume, and trabecular thickness in PBS-treated mice, wtRANKL-treated mice, and mtRANKL-immunized mice with wtRANKL injection (p<0.05).

FIG. 8A, 8B, 8C, 8D, 8E, 8F, and 8G show antiserum titers after immunization with mRANKL variants and the effect on osteoclastogenesis. In FIG. 8A and 8B, pearson's correlation coefficient was measured between antiserum titer and (A) CTX-1/RANKL or (B) BMD. All data are presented as the mean±SD of three independent measurements. Statistical differences were determined by one sample t-test.

FIG. 8C shows effect of sRANKL or mRANKL-MT3 induction on IFN-γ, IL-4, and IL-10 expression in splenic lymphocytes cells from sham or immunized mice. All data are presented as the mean±SD of three independent measurements. N.S., not significant (P>0.05), *P<0.05; **P<0.01.

FIG. 8D shows effects of dose-dependent antiserum titer immunization on the generation of TRAP-positive cells compared with control serum treatment. ALD treatment (10 μM) was used as a positive control. Representative TRAP staining (upper panel) and TRAP-positive multinucleated cell quantification (lower panel) in the presence of sRANKL (75 ng/mL).

FIG. 8E shows effect of dose-dependent antiserum titer immunization on bone resorption compared with control serum treatment. Error bars are mean±SD. *P<0.05 for sRANKL versus sRANKL+ALD, ††P<0.05 for sRANKL+Control Serum versus sRANKL+Immunized Serum and N.S., non-significant (P>0.05).

In FIG. 8F, TRAP, NFATc1, and OSCAR mRNA expression were analyzed by RT-PCR for anti-serum (1:1000)-treated BMMs in the presence of sRANKL compared to ALD treatment. Error bars are mean±SD. †P<0.05 for Non versus sRANKL+Control Serum, ††P<0.05 for sRANKL+Control Serum versus sRANKL+Immunized Serum, and *P<0.05 for sRANKL versus sRANKL+ALD.

FIG. 8G shows mRANKL variants immunization antiserum titers and its effects on osteoclastogenesis. The mRNA expression levels of ATP6vd2, ATP6v0a3, calcitonin receptor, Cathepsin K, c-fms, c-src, DC-STAMP, Integrin β3, MMP-9, RANK and LGR4 were analyzed by RT-PCR for each anti-serum (1:1000) treated BMMs in the presence of sRANKL compared to ALD treatment. All data are presented as the mean±SD of three measurements. †P<0.05 for Sham versus sRANKL+Control Serum group and ††P<0.05 for sRANKL+Control Serum group versus sRANKL+Immunized Serum and *p<0.05 for sRANKL+Control Serum group versus sRANKL+Sodium Alendronate (10 μM) treated group.

FIG. 9A shows a Micro-CT three-dimensional images of trabecular bone architectures of volume of interest (VOI) in tibias of a negative control, ovariectomized (OVX) mice, and OVX mtRANKL-immunized mice.

FIG. 9B shows bone mineral density, bone volume, % bone volume, and trabecular thickness in the negative control, OVX mice, and OVX mtRANKL-immunized mice (p<0.05).

FIG. 10A, 10B, 10C, 10D, 10E, 10F, 10G, 10H, 10I, 10J, and 10K show effect of anti-RANKL IgG induced by RANKL variants on sRANKL-induced mice femurs. FIG. 10A shows immunization and sampling schedule in sRANKL-induced mice.

In FIG. 10B, three-dimensional micro-CT images revealed the trabecular bone architecture of the volume of interest in Sham-, sRANKL with control IgG, and sRANKL with anti-RANKL mice femurs (n=10 images taken in total, one image for each mouse).

FIG. 10C shows bone mineral density (BMD), bone volume/trabecular volume (BV/TV), trabecular number (Tb. N.), and trabecular separation (Tb. Sp.).

FIG. 10D shows bone surface density (Bone surface/total volume), Cortical Bone area (Ct.Ar.), Cortical bone thickness (Ct.Th.) and Trabecular volume (TV); Error bars are mean±S.D.†P<0.05 for Sham versus sRANK+Control IgG, ††P<0.05 for sRANK+Control IgG versus sRANK+Anti−RANKL.

FIG. 10E shows histomorphometric analysis. Magnification is 20×. The scale bar represents 500 μm.

FIG. 10F shows TRAP staining (n=20 images taken in total, two images for each mousse) in the femurs. The arrow indicates TRAP positive cell. Magnification is 100×. The scale bar represents 50 μm.

FIG. 10G shows TRAP staining images of femurs. Magnifications are 20×. Size bar is 200 μm.

FIG. 10H shows parameters of femur osteoclasts. Oc.S/BS osteoclast surface per bone surface.

FIG. 10I shows Oc.N/BS, osteoclast number per bone surface.

FIG. 10J shows CTX-1 and 10K shows RANKL levels in mice sera. In FIG. 10C, H, I, J, K, Error bars are mean±SD. †P<0.05 for Sham versus sRANKL+Control IgG group and ††P<0.05 for sRANKL+Control IgG versus sRANKL+Anti-RANKL.

FIG. 11A, 11B, and 11C show effect of anti-RANKL treatment on sRANKL-induced osteoclastogenesis in BMMs. FIG. 11A shows effect of dose-dependent anti-RANKL immunization on TRAP-positive cell generation compared to control IgG treatment. Representative TRAP staining images (upper panel) and TRAP-positive multinucleated cell quantification (lower panel) in the presence of sRANKL (150 ng/mL).

FIG. 11B shows effect of dose-dependent anti-RANKL immunization on bone resorption compared with control IgG treatment. All data are presented as the mean±SD. N.S., not significant (P>0.05), **P<0.01 and ***P<0.001.

FIG. 11C shows the mRNA expressions of TRAP, NFATc1, OSCAR, ATP6vd2, ATP6v0a3, calcitonin receptor, Cathepsin K, c-fms, c-src, DC-STAMP, Integrin β3, MMP-9, RANK, and LGR4 were analyzed by RT-PCR of anti-RANKL IgG (0.5 μg/mL) treated BMMs compared with control IgG treatment (0.5 μg/mL) in the presence of sRANKL. Error bars are mean±SD. †P<0.05 for Non. versus sRANKL+Control IgG group and ††P<0.05 for sRANKL+Control IgG versus sRANKL+Anti-RANKL.

FIG. 12A and 12B show comparative inhibition of osteoclastogenesis by RANKL variants. FIG. 12A shows Co-IP for RANK- or LGR4-binding RANKL variants in BMMs. Each blot was obtained under the same experimental conditions.

FIG. 12B shows Western blots of RANK and LGR4 signaling pathway proteins. GAPDH was used as a loading control. The results are representative of three separate experiments with comparable results.

FIG. 13A, 13B, 13C, and 13D show effect of anti-RANKL treatment on sRANKL-induced signaling pathway in BMMs. FIG. 13A shows western Blot analysis. GAPDH was used as a loading control. Results are representative of three separate experiments with comparable results.

In FIG. 13B, NFATc1 nuclear translocation was analyzed by Western blot in cytosolic and nuclear fractions. Histone-H1 from the nuclear fraction or β-actin from the cytosol were used as loading controls. Densitometric analysis of NFATc1 in the cytosolic and nuclear fractions represents the mean ratio±SD of three separate experiments. Significant differences were seen at ***P<0.001 when comparing IgG with Anti-RANKL IgG.

FIG. 13C shows confocal microscopy images of NFATc1 nuclear translocation. Immunofluorescence images were acquired by staining for NFATc1 (green) and nuclei (blue). Magnification is 200×. The scale bar represents 20 μm.

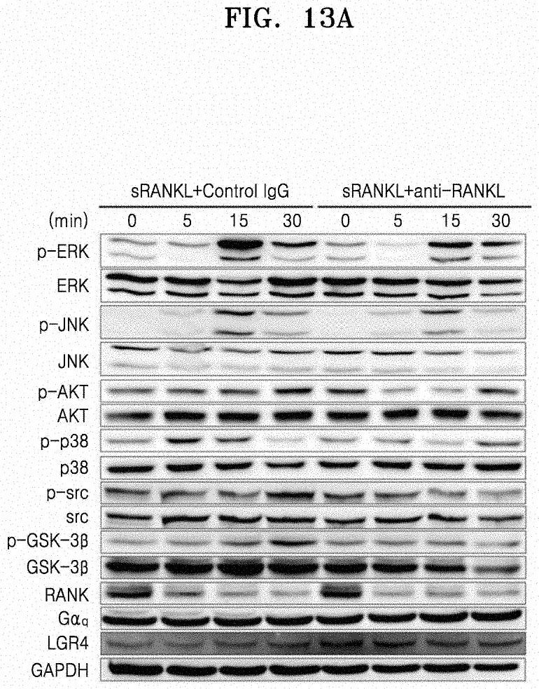

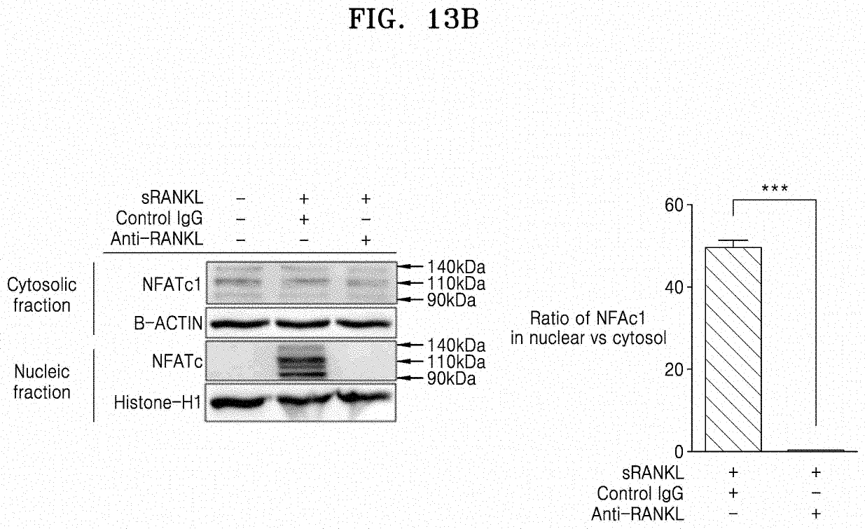

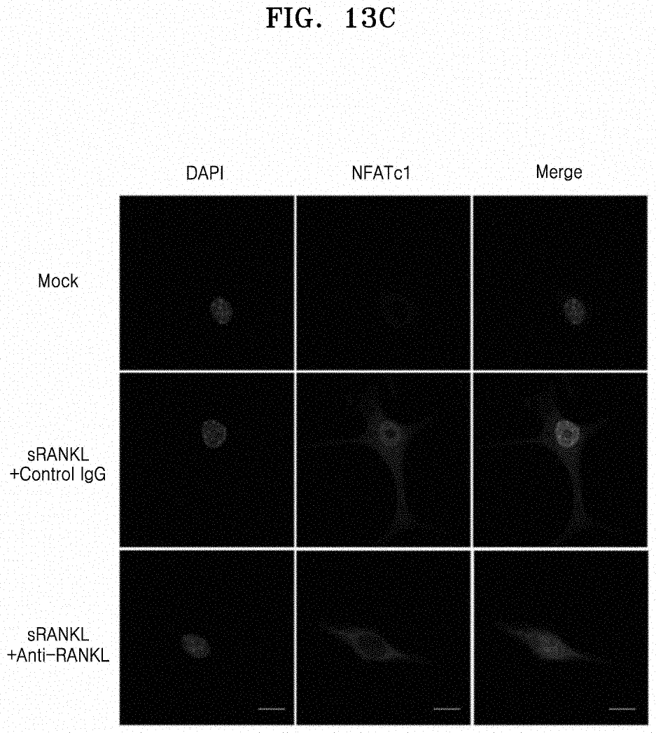

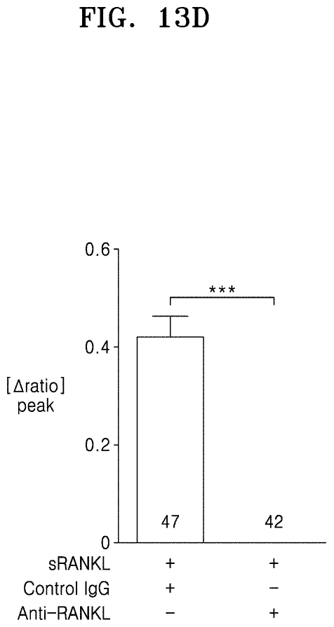

In FIG. 13D, intracellular calcium concentration ([Ca²⁺]i) was measured in BMMs. The numbers in x-axis means counted BMM. Data were expressed as mean±SD. ***P<0.001.

### MODE OF DISCLOSURE

Hereinafter, the present disclosure will be described in more detail with reference to exemplary embodiments. However, these exemplary embodiments are only for illustrating the present disclosure, and the scope of the present disclosure is not limited to these exemplary embodiments.

### REFERENCE EXAMPLE 1

### Preparation of RANKL, Mutagenesis, and Culture

1.1. Preparation of RANKL

RNA used for replication of RANKL cDNA was extracted from RANKL-expressing mouse MC3T3-E1 cells (Korean Cell line Bank, Seoul). The extracted RNA was identified by agarose gel electrophoresis. cDNA was prepared according to the manufacturer's instructions using an AccuPower RT PreMix Kit (Bioneer, Daejeon, Korea). A reaction mix included a Taq polymerase buffer, 10 mM dNTPs, 25 mM MgCl₂, 10 μM of primers (RANKL-K158: 5′-CAT ATG AAG CCT GAG GCC CAG CCA TT-3′, RANKL-D316: 5′-CTC GAG GTC TAT GTC CTG AAC TTT GAA AGC C-3′), 2.5 U of KOD DNA polymerase (EMD Millipore, Billerica, Mass., USA), and 2 μL of RANKL gene construct template, and amplification and replication of RANKL fragment were performed in the reaction mix.

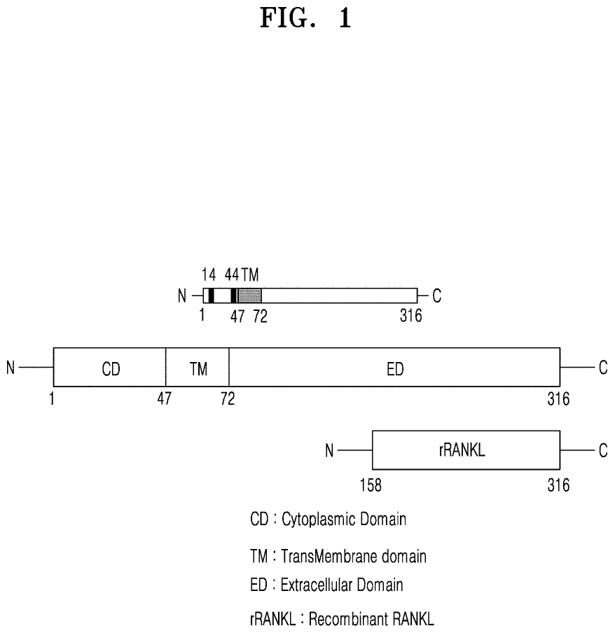

The thermal cycle consisting of a) initial denaturation at 95° C. for 5 minutes, b) denaturation at 95° C. for 30 seconds, c) annealing of primers at 55° C. for 30 seconds, and d) denaturation at 70° C. for 30 seconds was repeated a total of 40 cycles. The RANKL sequence encoded a full-length target region of 158 amino acids from 158 to 316 residues, as shown in FIG. 1.

1.2. Selection of RANKL Variants for Inhibition of Osteoclastogenesis

Amplification and cloning of the RANKL fragment and mutant RANKL candidates were carried out as mentioned in our previous study (Osteoporosis International volume 31, pages 983-993(2020)). The polymerase chain reaction (PCR) product was cloned into the Ndel/Xhol site in the pGEX-4T-1 vector (Promega, Madison, Wis., USA) and mutations at sites 180, 189-190, and 223-224 were introduced using megaprimers (Table 1). The PCR product was transformed into Escherichia coli BL21-Gold competent cells (Agilent, Santa Clara, Calif., USA) by electroporation (5 msec, 12.5 kV/cm). The transformed E. coli cells were cultivated in Luria-Bertani (LB) broth with ampicillin (50 μg/mL, T&I, Daejeon, Korea). The cloned product was confirmed by a commercial sequencing service (SolGent Co., Daejeon, Korea). All sequence data were analyzed using Vector NTI Advance 9.1.0 (Invitrogen, Carlsbad, Calif., USA).

TABLE 1
Megaprimers for site directed mutagenesis of mRANKL

| | | SEQ |
|---|---|---|
| Primer | Sequence | ID NO. |
| mRANKL-Ndel | 5′-CATATGAAGCCTGAGGCCCAGCCATTTGC-3′ | 5 |
| mRANKL-Xhol | 5′-CTCGAGGTCTATGTCCTGAACTTTGAAAGCC-3′ | 6 |
| mRANKL(K180R)-F | 5′-CCCATCGGGTTCCCATCGAGTCACTCTGTCCTCTTG-3′ | 7 |
| mRANKL(K180R)-R | 5′-CAAGAGGACAGAGTGACTCGATGGGAACCCGATGGG-3′ | 8 |
| mRANKL(D189I, | 5′-CTCTTGGTACCACATCAAGGGCTGGGCCAAGAT-3′ | 9 |
| R190K)-F | | |
| mRANKL(D189I, | 5′-ATCTTGGCCCAGCCCTTGATGTGGTACCAAGAG-3′ | 10 |
| R190K)-R | | |
| mRANKL-MT1 | 5′-AACATTTGCTTTCGGTTTTTTGAAACATCGGGAAGCG-3′ | 11 |
| (H223F, H224F)-F | | |
| mRANKL-MT1 | 5′-CGCTTCCCGATGTTTCAAAAAACCGAAAGCAAATGTT-3′ | 12 |
| (H223F, H224F)-R | | |
| mRANKL-MT2 | 5′-AACATTTGCTTTCGGTATTATGAAACATCGGGAAGCG-3′ | 13 |
| (H223Y, H224Y)-F | | |
| mRANKL-MT2 | 5′-CGCTTCCCGATGTTTCATAATACCGAAAGCAAATGTT-3′ | 14 |
| (H223Y, H224Y)-R | | |
| mRANKL-MT3 | 5′-AACATTTGCTTTCGGTTTTATGAAACATCGGGAAGCG-3′ | 15 |
| (H223F, H224Y)-F | | |
| mRANKL-MT3 | 5′-CGCTTCCCGATGTTTCATAAAACCGAAAGCAAATGTT-3′ | 16 |
| (H223F, H224Y)-R | | |
| mRANKL-MT4 | 5′-AACATTTGCTTTCGGTATTTTGAAACATCGGGAAGCG-3′ | 17 |
| (H223Y, H224F)-F | | |
| mRANKL-MT4 | 5′-CGCTTCCCGATGTTTCAAAATACCGAAAGCAAATGTT-3′ | 18 |
| (H223Y, H224F)-R | | |

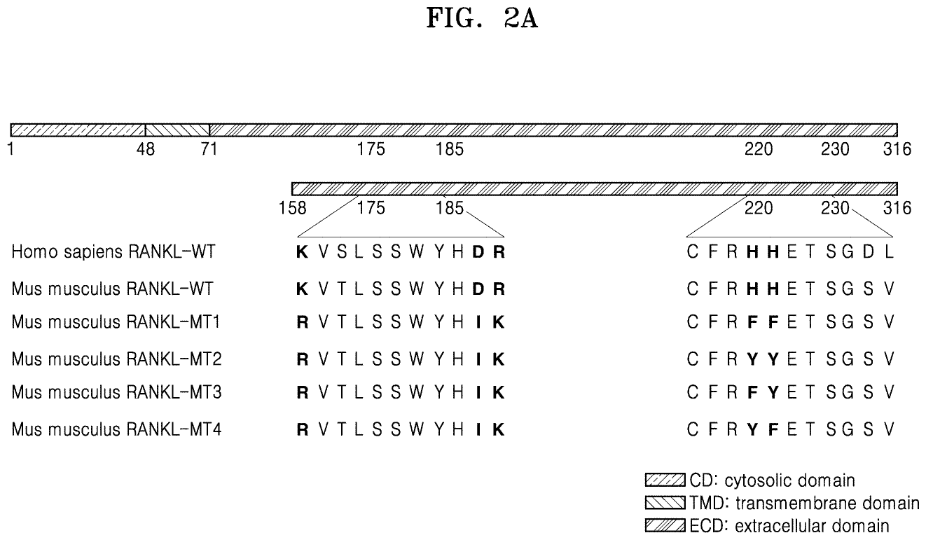

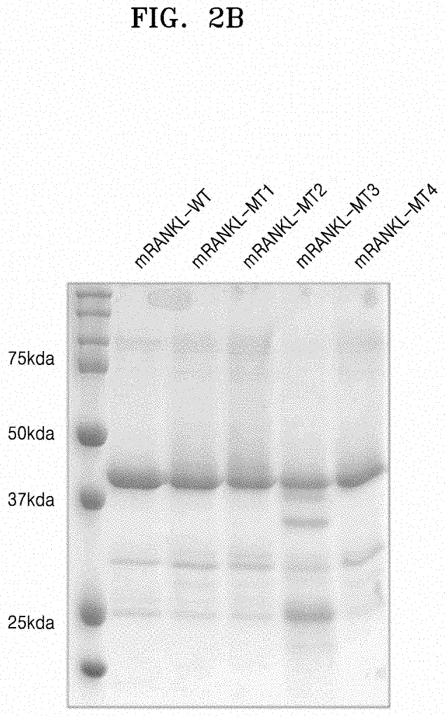

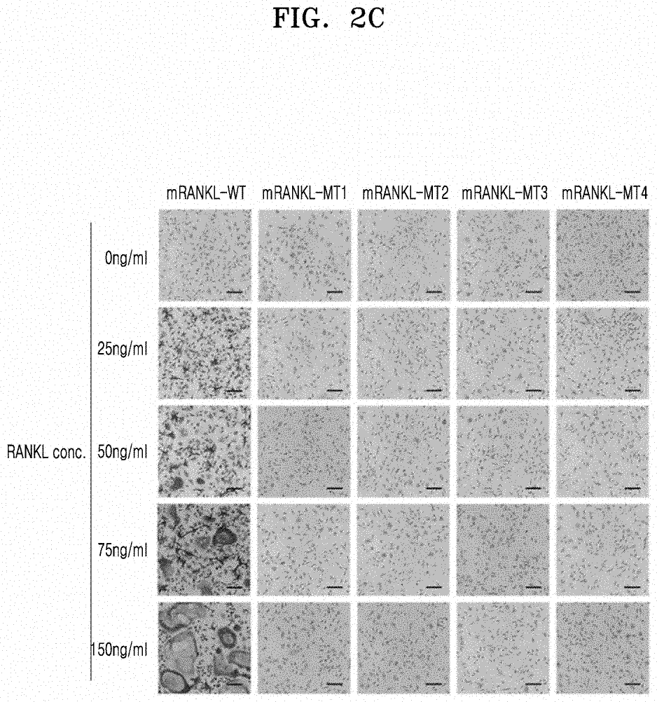

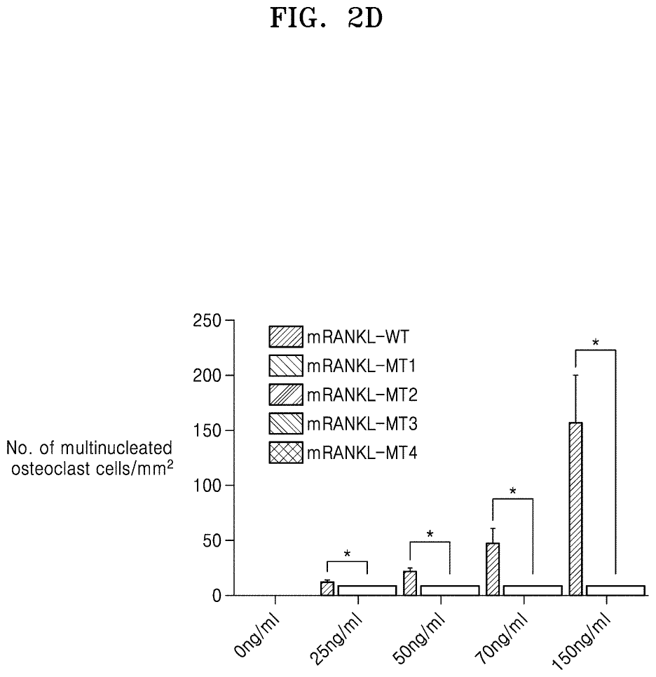

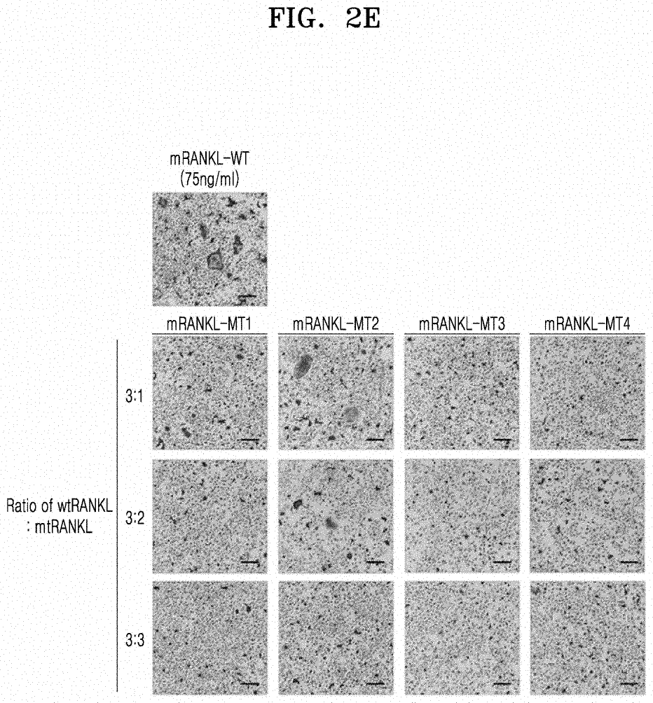

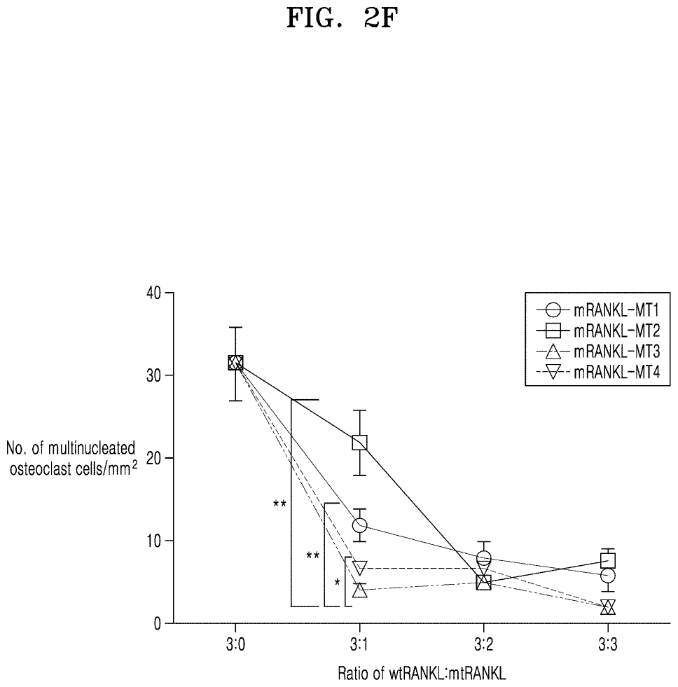

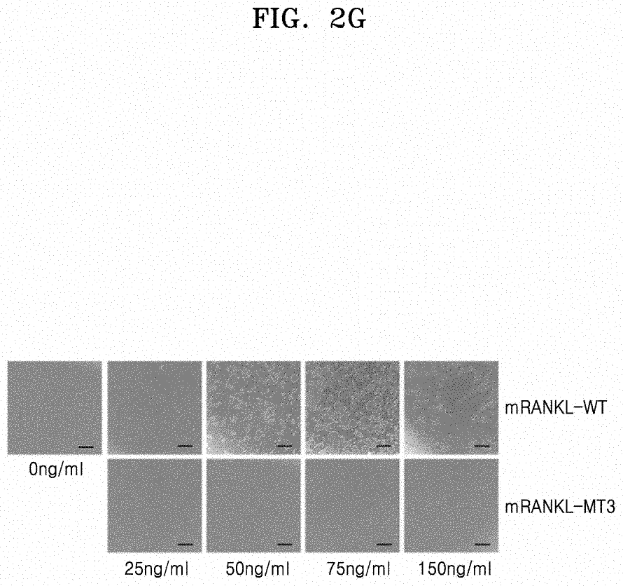

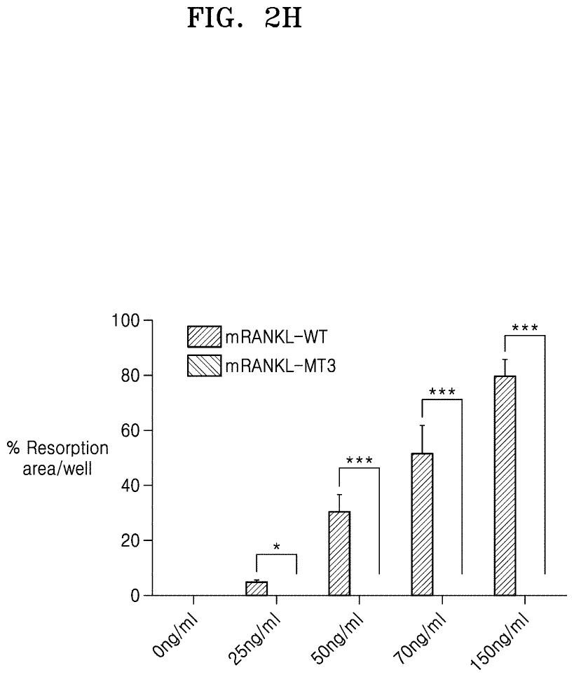

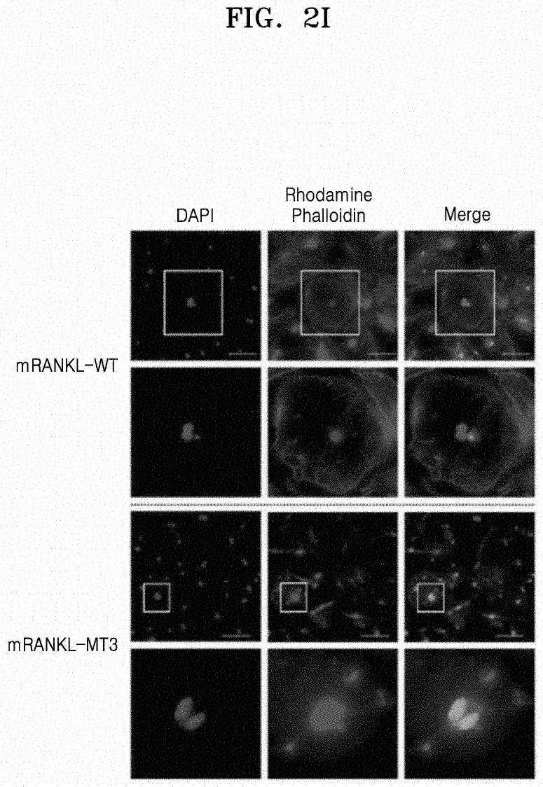

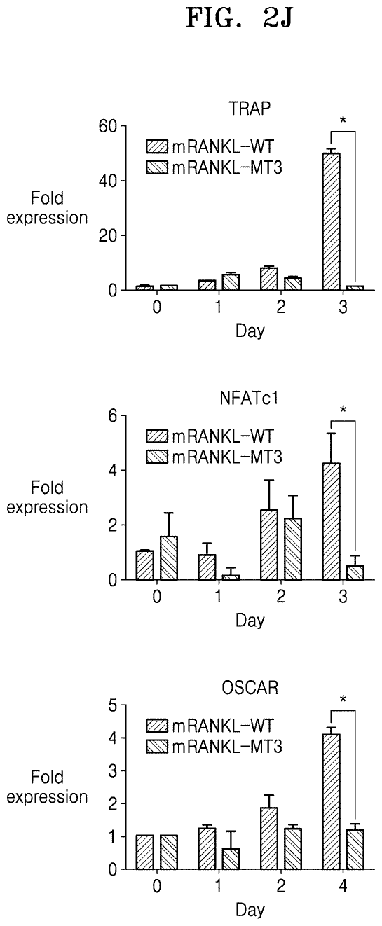

To find an optimal RANKL mutant that does not induce osteoclastogenesis, we selected four candidates with modified residues at the RANK binding site (FIG. 2A). The RANKL-RANK binding sites are K180, D189, R190, H223, H224, which are conserved between humans and mice. We selected 4 mutant RANKLs to be purified. Their size corresponded to the wild type RANKL (39 kDa) (FIG. 2B). Tartrate-resistant acid phosphate (TRAP) activity was absent for all RANKLs even at 150 ng/mL (FIG. 2C, D).

To investigate the optimized inhibitory effect against wild type RANKL, bone marrow-derived monocytes (BMMs) were treated with purified mutant and wild type RANKL (FIG. 2E, F). Among them, mRANKL-MT3 was the most effective at inhibiting TRAP activity, even at a 3:1 wild type:mutant ratio. Therefore, we used mRANKL-MT3 for subsequent experiments.

We used bone-resorption and F-actin ring formation assays to investigate osteoclast activity. To observe bone resorption in vitro, BMMs were cultured in Corning Osteo Assay Surface 96-well Multiple Well Plates (Sigma) with 30 ng/mL M-CSF and 75 ng/mL sRANKL (soluble RANKL; R&D Systems) or isolated RANKL for 6 days. Then, the plates were washed with pure water. To monitor actin ring formation, BMMs were grown on glass slides. After culture, the cells were fixed with 4% formalin, permeabilized with 0.5% Triton X100 in PBS for 5 min at room temperature, and incubated with 0.5 mg/mL TRITC-labelled phalloidin for 30 min. The cells were then rinsed with PBS. F-actin rings were visualized using a fluorescence ECLIPSE Ts2R microscope (Nikon, Tokyo, Japan).

In mature osteoclasts treated with wild-type RANKL, we observed numerous resorption pits and a ring of intracellular F-actin filaments in the sealing zone (FIG. 2G, 2H, 2I). However, with 150 ng/mL mRANKL-MT3 treatment, no resorption pits or F-actin rings were observed. TRAP, NFATc1, and OSCAR, mRNA expression, which is associated with osteoclastogenesis, was down-regulated in mRANKL-MT3-induced BMMs compared with to expression in mRANKL-WT induced BMMs (FIG. 2J).

Meanwhile, for Real-time PCR, BMM cells were incubated with 30 ng/mL M-CSF and 75 ng/mL sRANKL (R&D Systems) or isolated RANKL in 6-well plates. Total RNA was extracted using TRIzol reagent (Invitrogen, Thermo Fisher Scientific, Inc.). The cDNA was synthesized from 2 μg total RNA using ReverTra Ace qPCR RT Master Mix (TOYOBO, Osaka, Japan). Real-time PCR was conducted on a CFX Connect RealTime PCR Detection System (Bio-Rad, Hercules, Calif., USA) in a reaction mixture (total volume, 20 μL) containing IQ SYBR Green Supermix (Bio-Rad), 10 pmol forward primer, 10 pmol reverse primer, and 1 μg cDNA. The primer sequences used to target various genes are listed in Table 2.

TABLE 2

| | | Size | SEQ |
|---|---|---|---|
| Primer | Sequence | (bp) | ID NO. |
| TRAP-F | TAC CGT TGT GGA CAT GAC C | 150 | 19 |
| TRAP-R | CAG ATC CAT AGT GAA ACC GC | | 20 |
| OSCAR-F | CTG CTG GTA ACG GAT CAG CTC CCC | 310 | 21 |
| | AGA | | |
| OSCAR-R | CCA AGG AGC CAG AAC CTT CGA AAC T | | 22 |
| NFATc1-F | CAA CGC CCT GAC CAC CGA TAG | 392 | 23 |
| NFATc1-R | GGC TGC CTT CCG TCT CAT AGT | | 24 |
| Atp6v0d2-F | GAA GCT GTC AAC ATT GCA GA | 191 | 25 |
| Atp6v0d2-R | TCA CCG TGA TCC TTG CAG AAT | | 26 |
| c-fms-F | GCG ATG TGT GAG CAA TG CAG T | 341 | 27 |
| c-fms-R | GAG CCG TTT TGC GTA AGA CCT G | | 28 |
| ATP6v0a3-F | CGC CAC AGA AGA AAC ACT CA | 247 | 29 |
| ATP6v0a3-R | CCC AGA GAC GCA AGT AGG AG | | 30 |
| c-fos-F | ATG GGC TCT CCT GTC AAC AC | 336 | 31 |
| c-fos-R | GGC TGC CAA AAT AAA CTC CA | | 32 |
| MMP-9-F | TCC AGT ACC AAG ACA AAG | 183 | 33 |
| MMP-9-R | TTG CAC TGC ACG GTT GAA | | 34 |
| Cathepsin | TGT ATA ACG CCA CGG CAA A | 195 | 35 |
| K-F | | | |
| Cathepsin | GGT TCA CAT TAT CAC GGT CAC A | | 36 |
| K-R | | | |
| DC- | TGG AAG TTC ACT TGA AAC TAC GTG | 322 | 37 |
| STAMP(m)-F | | | |
| DC- | CTC GGT TTC CCG TCA GCC TCT CTC | | 38 |
| STAMP(m)-R | | | |
| Calcitonin | ACC GAC GAG CAA CGC CTA CGC | 272 | 39 |
| receptor-F | | | |
| Calcitonin | GCC TTC CAC GCC TTC AGG TAC | | 40 |
| receptor-R | | | |
| Integrin | TGA CTC GGA CTG GAC TGG CTA | 414 | 41 |
| β3-F | | | |
| Integrin | CAC TCA GGC TCT TCC ACC ACA | | 42 |
| β3-R | | | |
| RANK-F | CCA GGG GAC AAC GGA ATC A | 492 | 43 |
| RANK-R | GGC CGG TCC GTG TAC TCA TC | | 44 |
| LGR4-F | TAGGATTCAC TGGGACCCTA GTGCT | 160 | 45 |
| LGR4-R | CAGTTTGTGA AGATGAGCCA AGA | | 46 |
| β-actin-F | GTC CCT CAC CCT CCC AAA AG | 266 | 47 |
| β-actin-R | GCT GCC TCA ACA CCT CAA CCC | | 48 |

1.3. Mutagenesis and Transformation

A nucleotide sequence of a nucleic acid encoding amino acids substituted at positions 180, 189-190 and 223-224 of an amino acid sequence of mouse RANKL (mRANKL-MT3, hereinafter also referred to as “mtRANKL”) is represented by SEQ ID NO: 3. Mutagenesis of the gene was induced by megaprimers as described above. In detail, substitution of arginine for lysine at position 180, substitution of isoleucine for aspartic acid at position 189, substitution of lysine for arginine at position 190, substitution of phenylalanine for histidine at position 223, and substitution of tyrosine for histidine at position 224 in the amino acid sequence were performed.

Human RANKL protein (hRANKL) is 317 in total length, and contains one additional amino acid than the amino acid sequence of a mouse with a length of 316, and the RANKL substitution site is located one position downstream of the amino acid sequence of mouse. The amino acids at the substitution sites are identical to each other. A nucleotide sequence of a nucleic acid encoding such substituted amino acids (hRANKL-MT3) is represented by SEQ ID NO: 4.

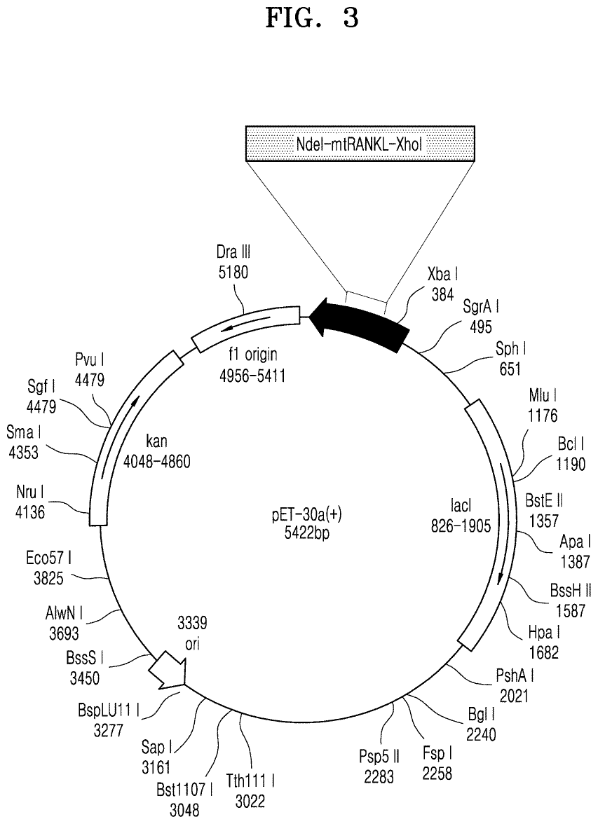

To replicate the resulting PCR product, the product was cloned into the Ndel/Xhol sites of a pET-30a vector (Novagen, Madison, Wis., USA) shown in FIG. 3. RANKL fragment was sub-cloned into pET30a, and for sequential translation of RANKL and 6xHis tags, it was confirmed to have the correct sequence. Transformation of the PCR product was performed using E. coli BL21-CodonPlus(DE3)-RIPL (Novagen) by electroporation (5 msec, 12.5 kV/cm). All sequencing was performed using a program of Vector NTI Advance 9.1.0 (Invitrogen, Carlsbad, Calif., USA).

1.4. Culture

A single colony containing the recombinant plasmid was inoculated into 20 mL of LB medium supplemented with kanamycin (50 μg/mL), and incubated at 37° C. under stirring at 200 rpm for 24 hours. 10 ml of this culture was inoculated into an Erlenmeyer flask containing 1 L of LB medium containing 50 μg/mL kanamycin. The culture was incubated at 37° C. with vigorous shaking at 180 rpm until the OD₆₀₀ value reached about 1.0. Subsequently, isopropyl β-D-1-thiogalactopyranoside (IPTG) was added at concentrations of 0 mM, 0.2 mM, 0.4 mM, 0.6 mM, 0.8 mM, and 1.0 mM to induce protein expression, followed by incubation for 6 hours. After induction, the culture was centrifuged at 5600 Yg at 4° C. for 20 minutes, and the cell pellet was stored at −20° C.

### REFERENCE EXAMPLE 2

### Purification of mtRANKL

After centrifuging the culture, the pelleted cells were resuspended in 10 mL of lysis buffer (20 mM sodium phosphate, 500 mM NaCl, 10 mM imidazole, pH 7.4). The cell suspension was supplemented with 0.1 mg/mL of lysozyme and 0.1 mM of phenylmethylsulfonyl fluoride (albiochem, La Jolla, Calif., USA), and incubated on ice for 1 hour. Then, glycerol (20% v/v, Carlo Erba, France) was added to the cell suspension. The cells were sonicated and centrifuged at 15,000 Yg for 10 minutes at 4° C. The supernatant was passed through a 0.2 μm filter paper, and then fixed to a Ni²⁺ affinity chromatography HisTrap FF column (1 mL, GE Healthcare Life Science, Piscataway, N.J., USA) equilibrated with a binding buffer (20 mM sodium phosphate, 500 mM NaCl, pH 7.4, 10 mM imidazole, 5 mM DTT, pH 7.4). Subsequently, the column was washed with a binding buffer supplemented with 20 mM imidazole. After washing, proteins were eluted with an elution buffer (Qiagen). The eluted proteins were dialyzed against a dialysis buffer (20% v/v glycerol in phosphate-buffered saline:PBS) in a 10,000 MW Slide-A-Lyzer dialysis cassette (Thermo Fisher Scientific, Waltham, Mass., USA). The purified proteins were concentrated under vacuum (Savant Instruments, Holbrook, N.Y., USA). Finally, the proteins were analyzed by sodium dodecyl sulfate polyacrylamide gel electrophoresis (SDS-PAGE), and the protein concentrations were calculated and determined by Bradford analysis. For endotoxin removal, an additional washing step was introduced after the initial washing for chromatography. In this step, W1-TX114 (W1-TX100 with 0.1% Triton X-114) or 1% sodium deoxycholate (buffer W1-DOC) was used at 80 times the volume of the resin bed, and the step was performed using W1-TX100 and W1-DOC at 25° C. or W1-TX114 at 4° C. The eluate from each washing step was collected for endotoxin quantification.

Recombinant protein samples were added to a 2× lysis buffer (0.5 M Tris-HCl, pH 6.8, 0.5% (/v) bromophenol blue, 10% (v/v) glycerol, 2% (v/v) SDS, and 10% (v/v) β-mercapto ethanol) at a ratio of 1:1 (v/v), and boiled for 5 minutes. Then, the proteins were analyzed by electrophoresis. After separation, the gel was stained with comassie brilliant blue G-250. To determine purity and recovery rate of the recombinant protein, the stained gel loaded with a predetermined amount of the protein was imaged at 300 dpi using a digital scanner (EPSON, USA).

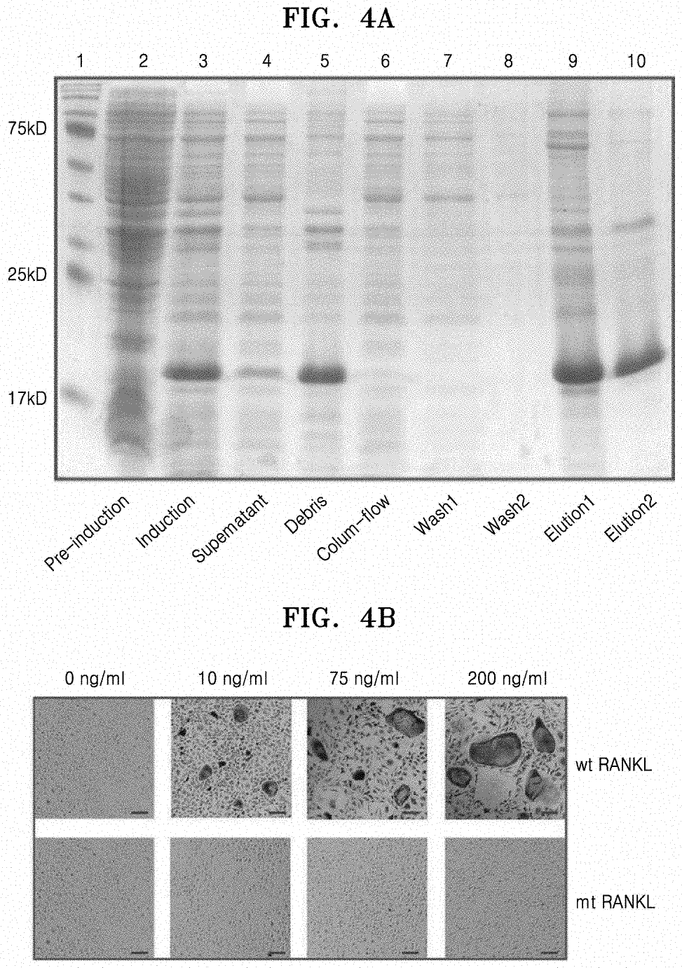

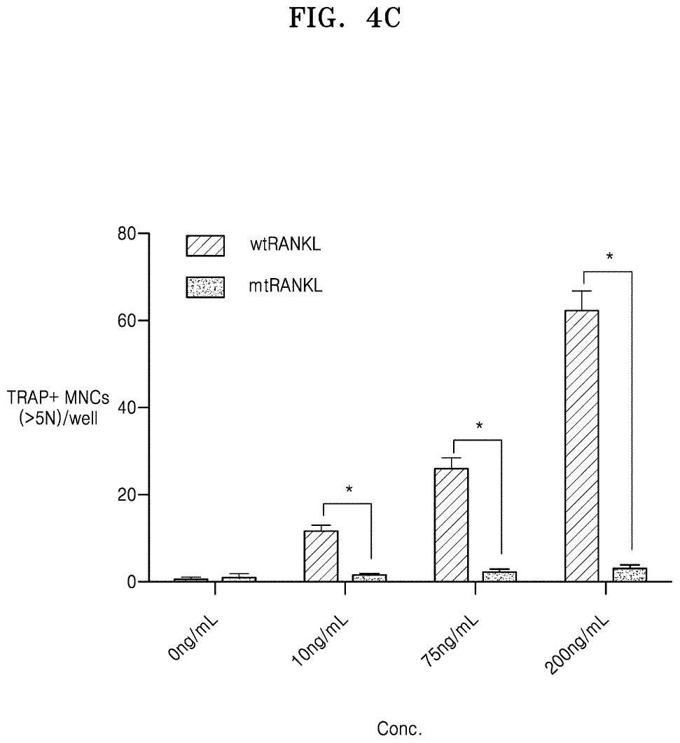

In FIG. 4A, SDS-PAGE showed a band of about 19 kDa in the whole cell protein extract of the induced sample.

### REFERENCE EXAMPLE 3

### TRAP Analysis

4-week old male BALB/c mice were purchased from Orient Bio (Gwangju, Korea) and kept in an animal facility approved by the Chosun University Animal Management Committee (IACUC2017-A0002). After obtaining bone marrow cells from mice, bone marrow mononuclear cells were seeded into 96-well plates (1 Y 10⁴ cells/well), and in the absence or presence of various concentrations of compounds, incubated with M-CSF (100 ng/mL) overnight before stimulation with RANKL (50 ng/well). The medium was replaced every other days. 6 days later, the cells were fixed with 4% paraformaldehyde, permeated in 0.1% Triton X-100, and washed with PBS. Then, TRAP activity (Sigma-Aldrich, St. Louis, Mo., USA) staining was performed. TRAP-positive multinuclear cells containing 5 or more nuclei were counted as osteoclasts.

### REFERENCE EXAMPLE 4

### Western Blotting

Proteins were separated by SDS-PAGE and electropermeated into a nitrocellulose membrane (Bio-Rad). After blocking the membrane with 5% (weight/volume) skim milk in TBST [10 mM Tris (pH 7.5), 150 mM NaCl, 0.1% (vol/vol) Tween 20], each mouse serum for primary antibody in the blocking solution was examined. The membrane was washed three times with Tris buffered saline. Horse peroxidase (HRP)-conjugated secondary antibody was diluted 1:5000 with TBST in 1% (wt/vol) skim milk powder. ECL_system (Amersham Pharmacia Biotech) was used as the membrane.

Primary antibodies: AKT (Cell Signaling Technology, 1:1000), phospho-AKT (Cell Signaling Technology, #9271S, 1:1000), p38 (Cell Signaling Technology, 9212S, 1:1000), phospho-p38 (Cell Signaling Technology, #9211S, 1:1000), ERK (Cell Signaling Technology, 9102S, 1:1000), phospho-ERK (Cell Signaling Technology, #9101S, 1:1000), JNK (Cell Signaling Technology, 9252S, 1:1000), phosphoJNK (Cell Signaling Technology, #9251S, 1:1000), GSK-3β (Cell Signaling Technology, 9315S, 1:1000), phosphoGSK-3β (Cell Signaling Technology, #9336S, 1:1000), Src (Cell Signaling Technology, 2108S, 1:1000), phosphoSrc (Cell Signaling Technology, #2105S, 1:1000), NF-KB p65 (Cell Signaling Technology, #3034, 1:1000), phosphop65 (Cell Signaling Technology, #3031, 1:1000), RANK (Cell Signaling Technology, 4845S, 1:1000), Gaq (Cell Signaling Technology, 14373S, 1:1000), LGR4 (MyBioSource, MBS468030, 1:500), 20112360C-1, and GAPDH (Santa-Cruz Biotechnology, 1:2500).

### REFERENCE EXAMPLE 5

### Enzyme-Linked Immunosorbent Assay (ELISA)

A commercially available enzyme immunoassay kit (R & D Systems, Minn., USA) was used to measure RANKL levels in the serum of mice according to the manufacturer's protocol. Absorbance (450 nm) was measured in a colorimetric microplate reader (BioTek, Winooski, USA). The reading of the absorbance was then subtracted from the reading at 570 nm.

### REFERENCE EXAMPLE 6

### Micro-Computed Tomography (Micro-CT)

The right femur of each mouse subjected to each experiment was incised and CT imaging was performed using a Quantum GX pCT imaging system (PerkinElmer, Hopkinton, Mass., USA) located at the Korea Basic Science Institute in Gwangju. As for the X-ray source, the field of view was set to 45 mm, 90 kV and 88 mA (voxel size, 90 μm; scanning time, 14 minutes). CT imaging was performed using a Quantum GX 3D Viewer software. After scanning, image segmentation was performed in Analyze (AnalyzeDirect, Overland Park, Kans., USA). Briefly, the legs were segmented using semi-automatic and manual tools (e.g., object extractor, area expansion, and object separator) using volume editing tools. Then, a 3D rendering of the leg was created, and bone mineral density (BMD), bone volume (BV), bone volume percentage (%), and trabecular thickness were calculated using the ROI tools.

### REFERENCE EXAMPLE 7

### Statistical Analysis

For statistical analysis, a two-way paired Student's t test was used. P<0.05 was considered statistically significant. Data were expressed as mean±standard deviation (SD) unless otherwise specified. Data were analyzed using an SPSS version 20.0 software program for Windows (SPSS, Chicago, Ill., USA). A GraphPad Prism version 6.00 software program for Windows (GraphPad, La Jolla, Calif., USA) was used to analyze data in vitro and in vivo experiments, and the data was expressed as mean±SD.

### EXAMPLE 1

### In Vitro Assay of Osteoclastogenesis

To evaluate the effect of mtRANKL on osteoclastogenesis, TRAP analysis was performed on RANKL-treated primary bone marrow-derived macrophages (BMM) by the method described in Reference Example 2, and the results are shown in FIGS. 4B and 4C.

As shown in FIGS. 4B and 4C, BMM differentiated into mature TRAP-positive multinuclear osteoclasts, and the number thereof was significantly higher in wild-type RANKL (wtRANKL)-treated cells than in mtRANKL-treated control cells. Therefore, it was found that mtRANKL did not induce osteoclast differentiation, unlike wtRANKL.

### EXAMPLE 2

### Assay of Bone destruction of mtRANKL

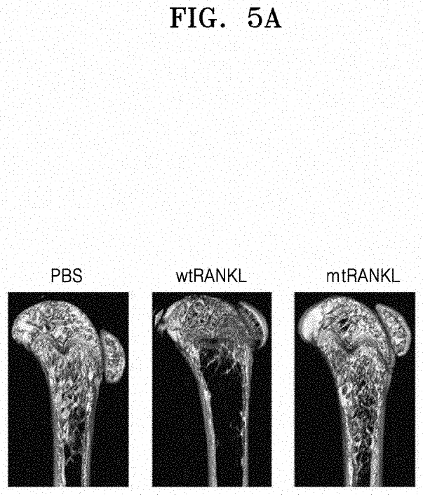

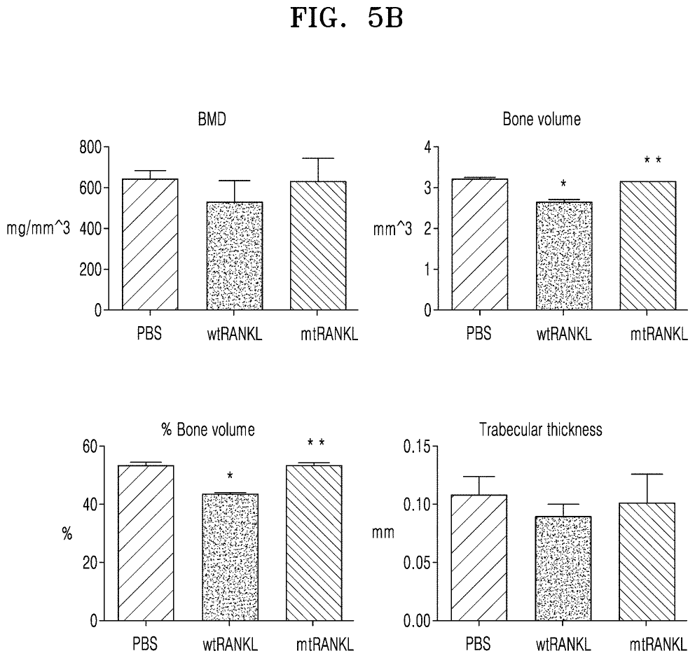

To investigate the bone-destructive effect of mtRANKL, 4-week-old male BALB/c mice (Orient Bio) were placed in a cage capable of accommodating up to 4 animals in a light-controlled environment (2-hr light-dark cycle) and provided with autoclaved water and both whole and powdered rodent diet (Purina, St. Louis, Mo., USA). Starting from 6 weeks of age, 9 mice were randomly divided into three groups, and each group was subcutaneously injected with wild-type RANKL (Amgen, Thousand Oaks, Calif., USA), mtRANKL purified in Reference Example 2, and saline three times a week (1.0 mg/kg). The trabecular bone in femoral neck was examined by Micro-CT analysis described in Reference Example 6. The results are shown in FIGS. 5A and 5B.

As shown in FIG. 5A, mild osteoporosis was observed in the wtRANKL-injected mouse. BMD, bone volume (BV), percentage (%) of bone volume, and trabecular thickness were remarkably reduced. However, as shown in FIG. 5B, no significant change was observed in the trabecular bone of the mtRANKL-injected mouse. Therefore, it is determined that mtRANKL had no effect on osteoclast differentiation.

### EXAMPLE 3

### Analysis of Effect of mtRANKL Immunization

Since mtRANKL has no osteoclast-stimulating activity, immunization with mtRANKL may induce anti-RANKL antibodies that would inhibit the osteoclast-stimulating activity of exogenous RANKL. To demonstrate this possibility, a wtRANKL-induced mouse was used as an osteoporosis animal model to examine the therapeutic effect of mtRANKL protein immunization on osteoporosis.

3.1. mtRANKL Immunization

17-week-old male BALB/c mice were equally divided into 3 groups. The group was divided into a negative control group in which PBS was intraperitoneally injected, a group in which only wtRANKL was injected, and a group in which wtRANKL was injected after immunization with mtRANKL for 52 days. mtRANKL was injected after mixing with an aluminum hydroxide adjuvant. The immunization process started with 1^{st} immunization of mtRANKL at 52 days, 2^{nd} immunization at 39 days, 3^{rd} immunization at 14 days each before injection of wtRANKL, and a total of 3 immunizations was performed. The amount of mtRANKL was 0.2 mg per mouse.

After 52 days, PBS was injected into the negative control group and wtRANKL was injected into the other two groups. Next day, wtRANKL was injected once more. wtRANK was subcutaneously injected (2.0 mg/kg). Serum was collected to determine the titer of anti-RANKL antibody. Then, after extracting tibias, the adhesive soft tissues of tibias were removed.

3.2. Result of Antibody Production

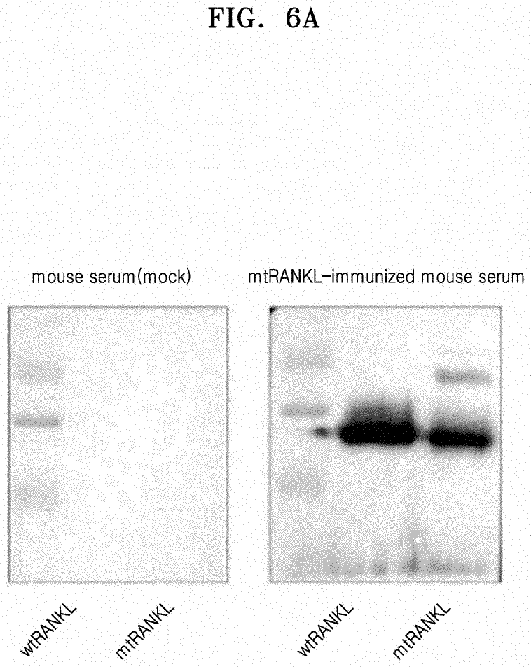

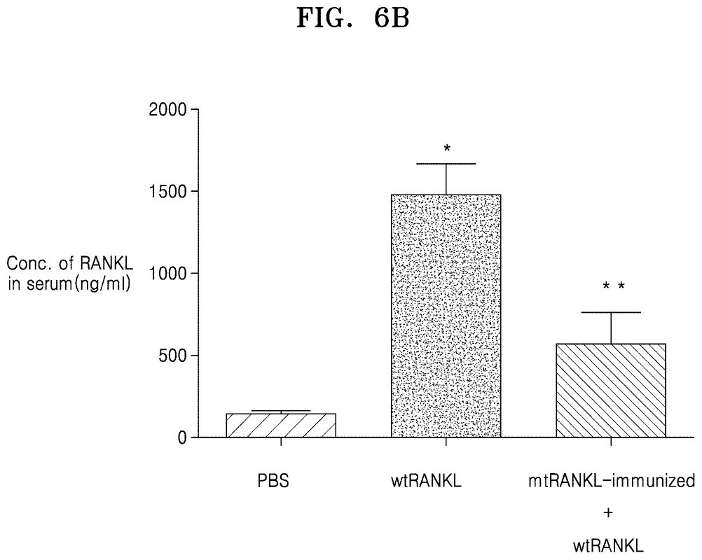

To determine whether antibodies were produced due to mtRANKL immunization, Western blot of Reference Example 4 was performed on the collected serum, and the results are shown in FIG. 6A.

As shown in FIG. 6A, antibodies were detected in the serum of mice immunized with mtRANKL. In contrast, no antibodies were detected in non-immunized mice (mocks).

In addition, ELISA analysis was performed by the method described in Reference Example 5, and the results are shown in FIG. 6B.

As shown in FIG. 6B, when wtRANKL was administered to non-immunized mice, the level of RANKL in the blood was increased. In contrast, when wtRANKL was administered to mice immunized with mtRANKL, RANKL in blood was eliminated, as compared with non-treated mice. Accordingly, it was found that the anti-RANKL antibodies were produced in the mouse body due to the immunization with mtRANKL.

3.3. Analysis Results of Bone Destruction according to Immunization

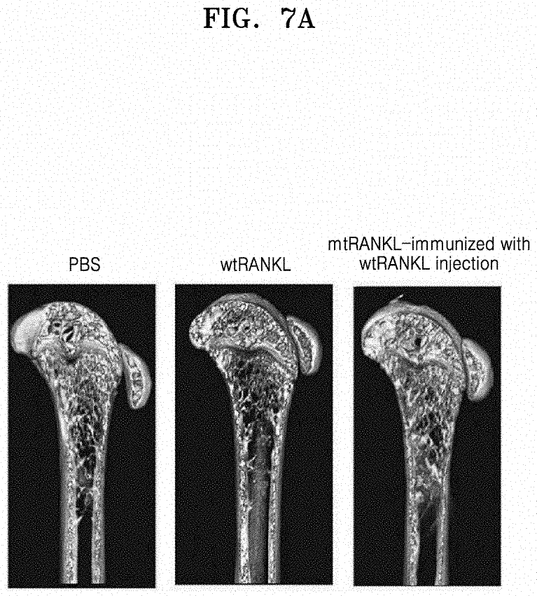

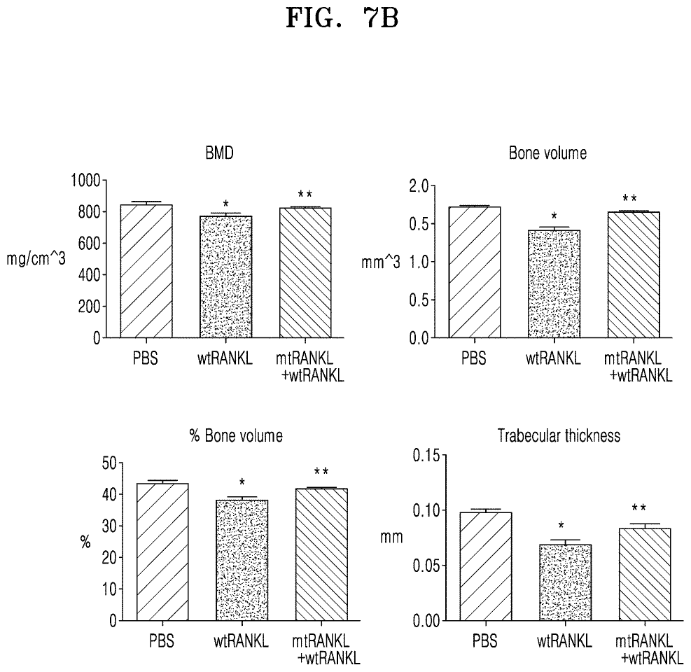

To examine whether RANKL has a bone destruction effect when RANKL was injected after immunization with mtRANKL, micro-CT analysis was performed by the method of Reference Example 6. Representative three-dimensional images and graphs of the proximal tibias are shown in FIGS. 7A and 7B.

As shown in FIG. 7A, the wtRANKL-injected mouse had fenestrated trabecular bone and thinner rods forming the trabecular bone, as compared with the PBS-treated mouse. In addition, mild osteoporosis was observed. However, like the PBS-treated mice, the trabecular bones of mtRANKL-immunized mice with wtRANKL injection had thicker fibers and higher density than the wtRANKL-injected mice, which were similar to those of the PBS-treated mice.

As shown in 7B, BMD (821.1±14.8 mg/cm³, P<0.05) of tibias of PBS-treated mice and BMD (795.8±11.9 mg/cm³, P <0.05) of tibias of mtRANKL-immunized mice with wtRANKL injection were significantly higher than BMD (738.0±19.1 mg/cm³, P<0.05) of tibias of wtRANKL-injected mice.

In addition, the values of the bone volume and percentage of bone volume of PBS-treated mice and mtRANKL-immunized mice with wtRANKL injection were maintained at similar levels, and the values of wtRANKL-injected mice were decreased. Accordingly, it was found that the immunization with mtRANKL resulted in the production of anti-RANKL antibodies in the mouse body, leading to reduction of the induction of osteoclast differentiation.

3.4. Antiserum Titers

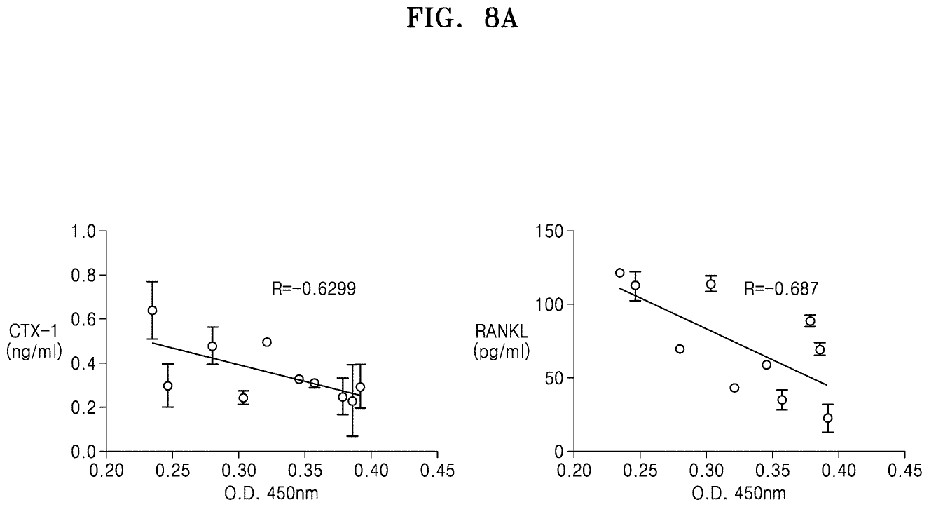

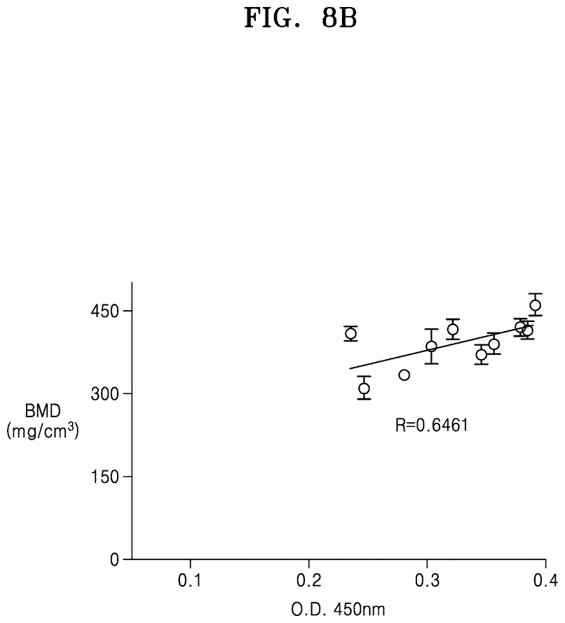

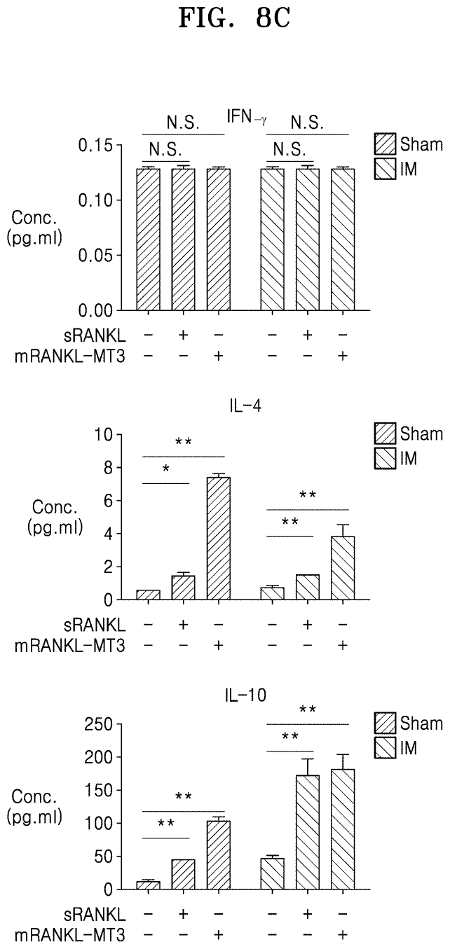

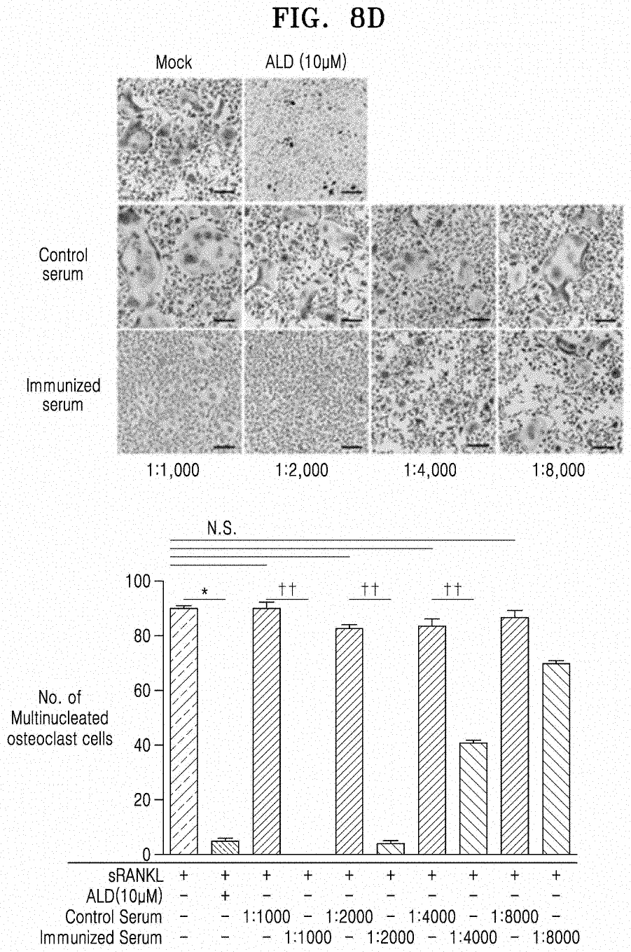

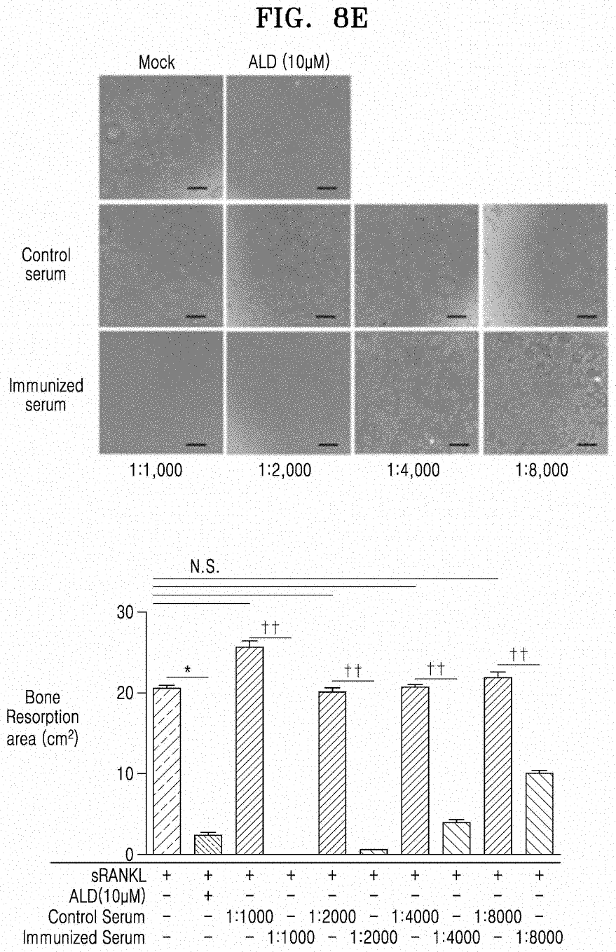

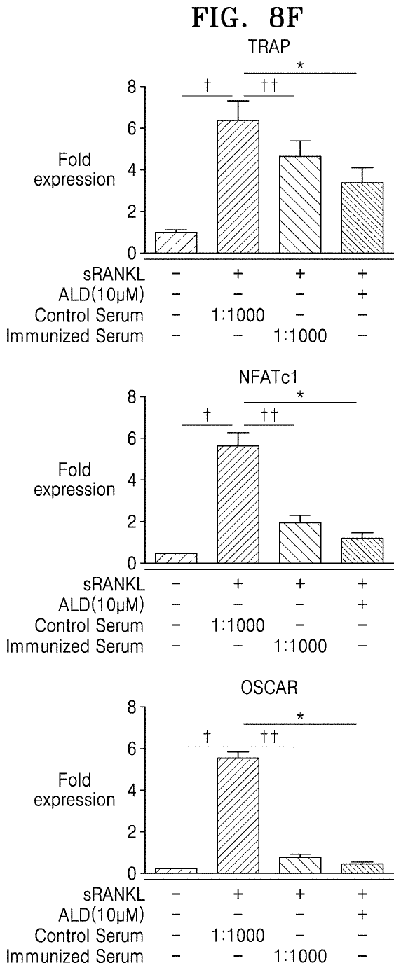

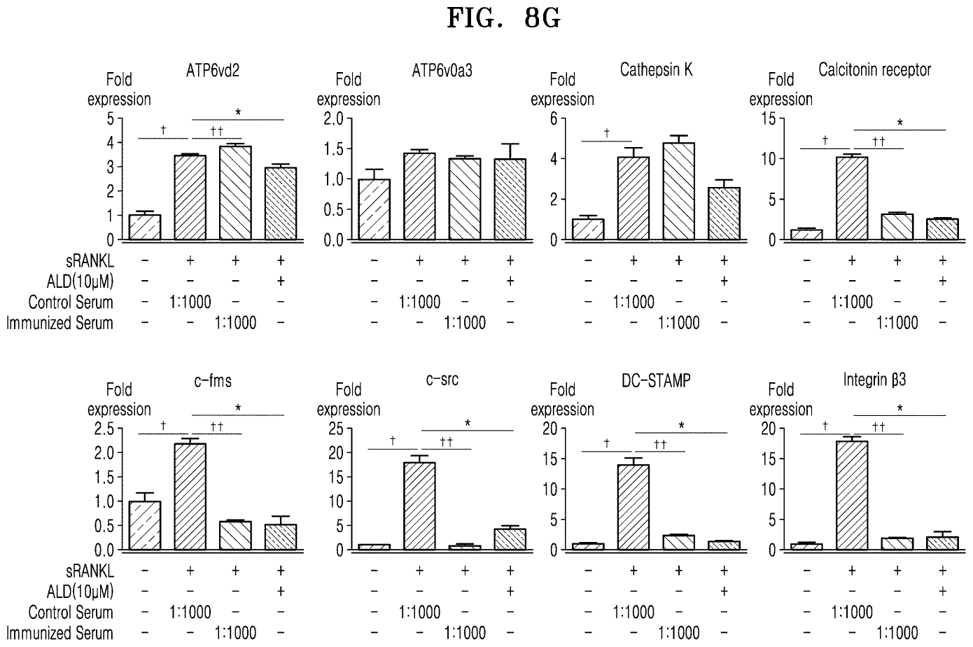

To investigate the effect of anti-mRANKL IgG antibodies on bone remodeling markers and soluble RANKL in the serum, relationships with anti-RANKL IgG antibodies and CTX-1/RANKL were assessed in the OVX+IM group. The generation of anti-mRANKL IgG antibodies was negatively correlated with CTX-1 (R=−0.6299, P=0.0509, FIG. 8A, left) and sRANKL (R=−0.687, P=0.0282, FIG. 8A, right) levels in mouse serum. However, we observed a positive linear correlation between BMD and anti-RANKL IgG antibodies (R=0.6461, P=0.0027, FIG. 8B).

To determine whether mRANKL-MT3 immunization influences Th1 and Th2 cytokine production, we evaluated the effects of mRANKL-MT3 on IL-4 and IL-10 secretion, which are markers for Th2 responses, and IFN-γ, a marker for Th1 responses, in the culture supernatant of isolated spleen cells stimulated with sRANKL or mRANKL-MT3 (FIG. 8C). In Sham and IM-stimulated splenocytes, there were no significant differences in IFN-γ levels in the presence of sRANKL or mRANKL-MT3. However, IL-4 and IL-10 secretion significantly increased due to sRANKL or mRANKL-MT3 treatment in Sham and IM-stimulated splenocytes, suggesting that anti-RANKL production by mRANKL-MT3 vaccination is Th2-B cell-mediated.

To examine antiserum effects on osteoclastogenesis, we treated primary BMMs with antiserum obtained from PBS- or mRANKL-MT3-immunized mice. While antiserum obtained from PBS-immunized mice showed no effect on inhibition of osteoclastogenesis, the formation of TRAP-positive multinucleated cells was significantly decreased by mRANKL-MT3-immunization-induced antiserum, even at a dose of 1:4000 (FIG. 8D). In addition, antiserum from mRANKL-MT3-immunized mice significantly inhibited bone resorption activity (FIG. 8E). Notably, antiserum from mRANKL-MT3-immunized mice caused a slight decrease in NFATc1 and significantly downregulated TRAP, OSCAR, and other osteoclastogenic mRNA (FIG. 8F, 8G).

### EXAMPLE 4

### Ovariectomy (OVX) Analysis

4.1. Immunization and Ovariectomy (OVX)

17-week-old male BALB/c mice were equally divided into a group that was given OVX after immunization with mtRANKL, a group that was given only OVX, and a non-treated negative control group.

The mtRANKL-immunized group was immunized with mtRANKL a total of three times: started with 1^{st} immunization at 52 days, 2^{nd} immunization at 39 days, 3^{rd} immunization at 14 days each before OVX. mtRANKL used in the immunization was injected after mixing with an aluminum hydroxide adjuvant. The amount of mtRANKL was 0.2 mg per mouse.

52 days after 1^{st} immunization, OVX was performed. On day 70 after OVX, serum were collected and tibias were extracted, and then the adhesive soft tissues of tibias were removed.

4.2. Result of Analysis of Effect

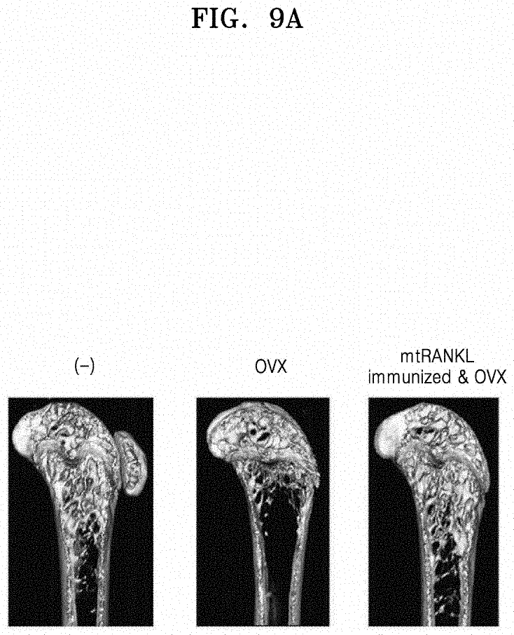

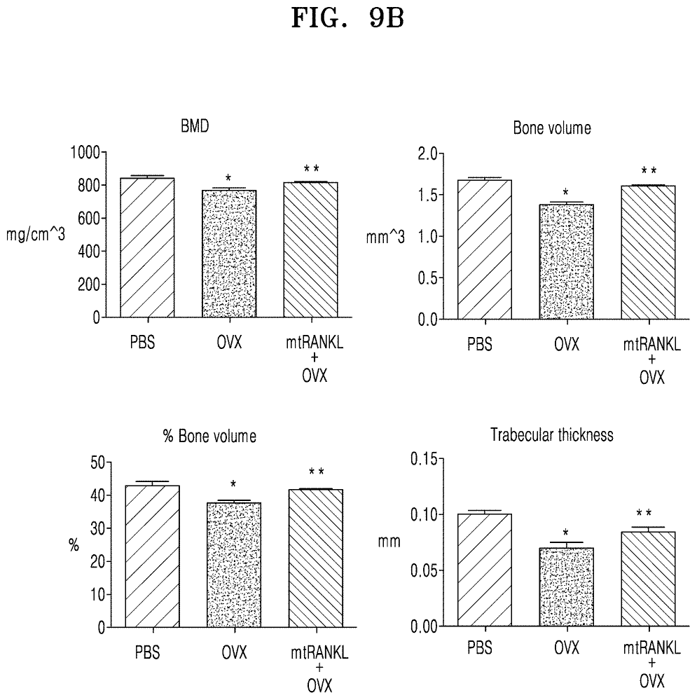

To analyze the mtRANKL immunization effect on the ovariectomized mice (OVX), micro-CT analysis according to Reference Example 6 was performed. Representative three-dimensional images of the proximal tibias and graphs showing values of BMD, bone volume, percentage of bone volume, and trabecular thickness are shown in FIGS. 9A and 9B, respectively.

As shown in FIG. 9A, the trabecular bones of the OVX mice showed mild osteoporosis, as compared with that of the non-OVX mice. However, trabecular bones of OVX mice ovariectomized after immunization with mtRANKL had thicker fibers and higher density than those of OVX mice, and were similar to those of the negative control mice (−).

As shown in FIG. 9B, BMD of tibias of mice ovariectomized after immunization with mtRANKL and BMD of the negative control group were significantly higher than that of the OVX mice. Similarly, the mtRANKL-immunized mouse group had similar values to the negative control group in terms of the bone volume with respect to the total volume, percentage (%) of bone volume, and trabecular thickness. These results show that immunization with mtRANKL may also block the induction of osteoclast differentiation in OVX mice given ovariectomy.

### EXAMPLE 5

### Effect of Anti-RANKL IgGs in sRANKL-Induced Mice

We further evaluated the effect of anti-RANKL IgGs obtained from mRANKLMT3-immunized mouse sera on bone metabolism and osteoclastogenesis. Anti-RANKL IgGs were purified with a protein G column and confirmed by immunoblotting with sRANKL.

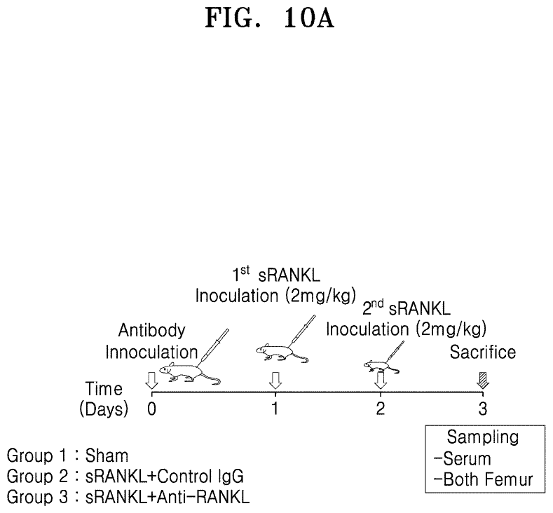

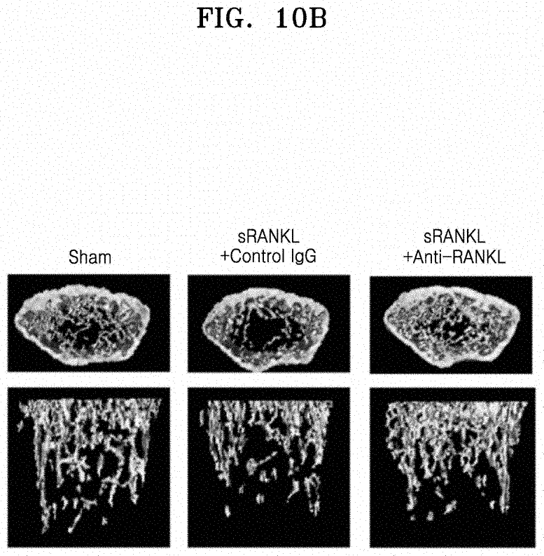

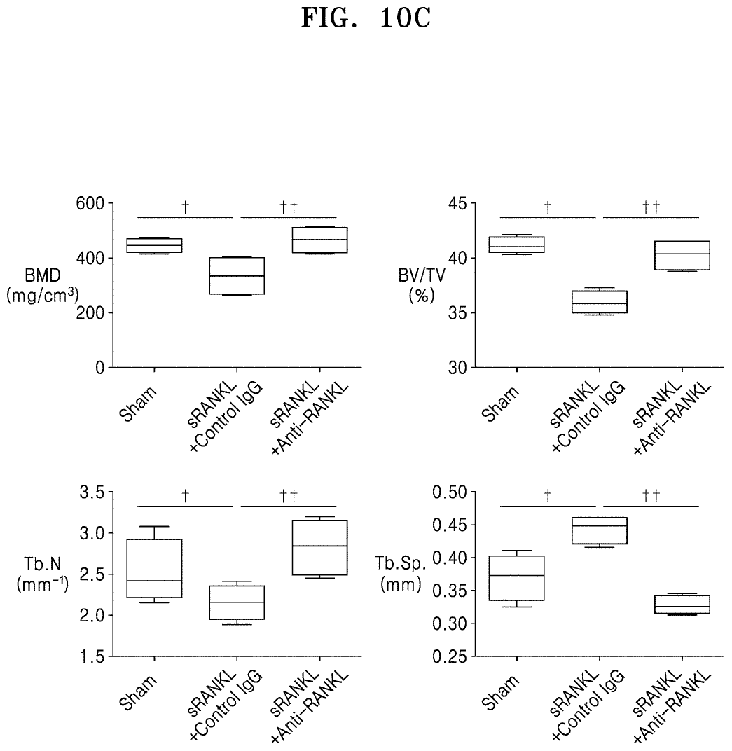

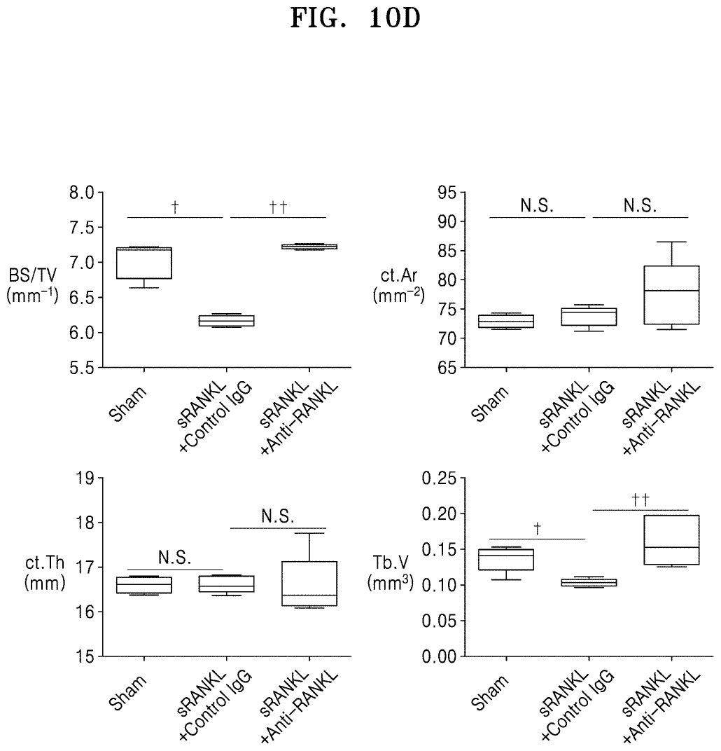

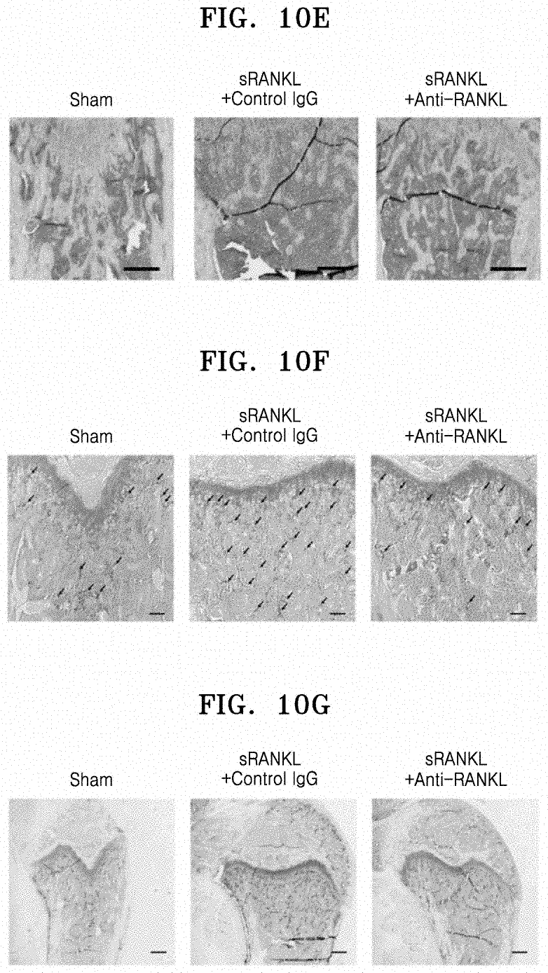

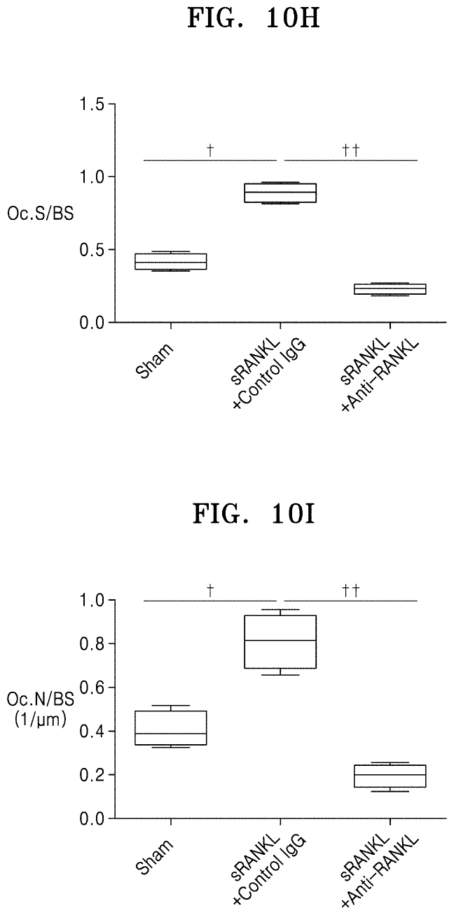

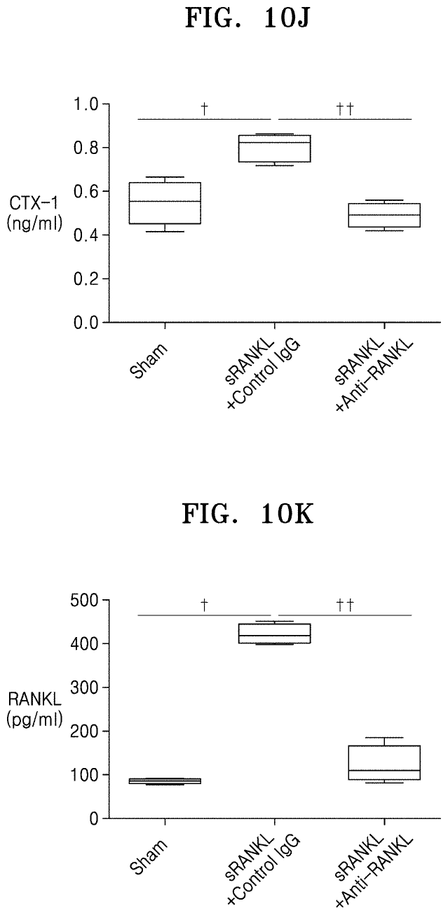

To investigate the effect of anti-RANKL on osteolysis inhibition, purified anti-RANKL from immunized mouse sera were inoculated in sRANKL-treated mice (FIG. 10A). As shown in the micro-CT scan results, sRANKL-treated mice showed fenestrated plate-like structures of the trabecular bone and thinner rods forming the trabecular network, which finally degraded and left the structure less connected, indicating mild osteoporosis (FIG. 10B). However, purified anti-RANKL treatment markedly improved the trabecular bone architecture of VOI extracted from the distal femur in sRANKL-induced mice, which was reduced in osteoporosis. Aside from visual assessment, BMD, BV/TV, Tb.N., Tb. Sp, and other bone scores were evaluated by quantitative micro-CT (FIG. 10C, 10D). As expected, low BMD, BV/TV, and Tb.N. values in sRANKL-induced mice were significantly rescued by anti-RANKL treatment. These results clearly demonstrate the therapeutic effects of anti-RANKL as an active immunogen for osteoporosis. Bone histomorphometric analysis and TRAP staining of mice femur were performed, revealing a fragmented network of the trabecular bone in sRANKL-treated mice and an increased number of TRAPpositive osteoclasts (FIG. 10E, 10F, 10G). However, a dense network with minimal spaces and fewer TRAP-positive cells were observed in the presence of antiRANKL. These results confirmed that the OCs/BS % and OCs/mm² values in sRANKL-induced mice were significantly decreased after anti-RANKL treatment (FIG. 10H, 10I).

Also, circulating sRANKL and CTX-1 levels were higher in sRANKL-treated mice, while anti-RANKL treatment significantly decreased circulating RANKL and CTX-1 levels (FIG. 10J, 10K).

### EXAMPLE 6

### Effect of Anti-RANKL on sRANKL-Induced Osteoclastogenesis In Vitro

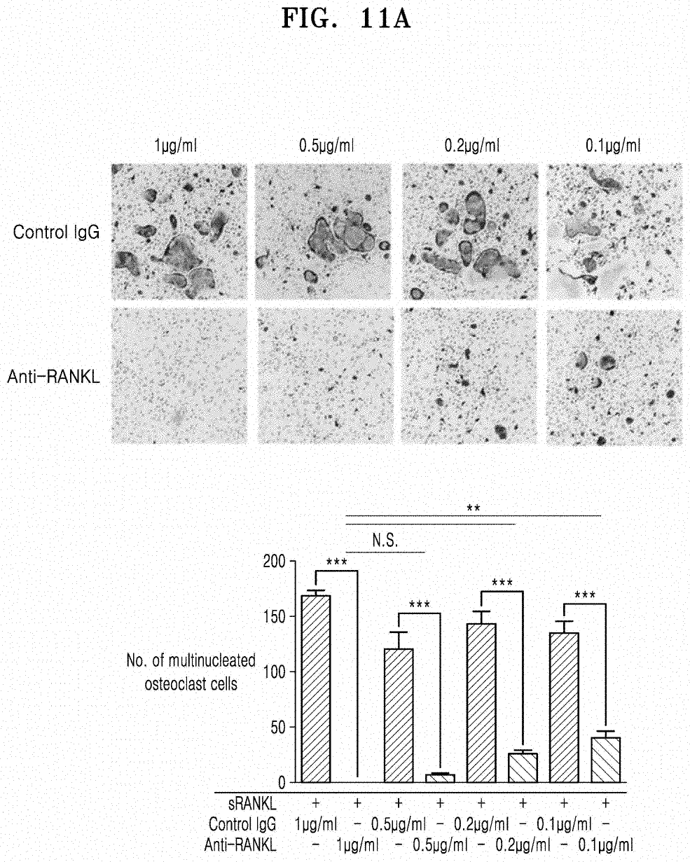

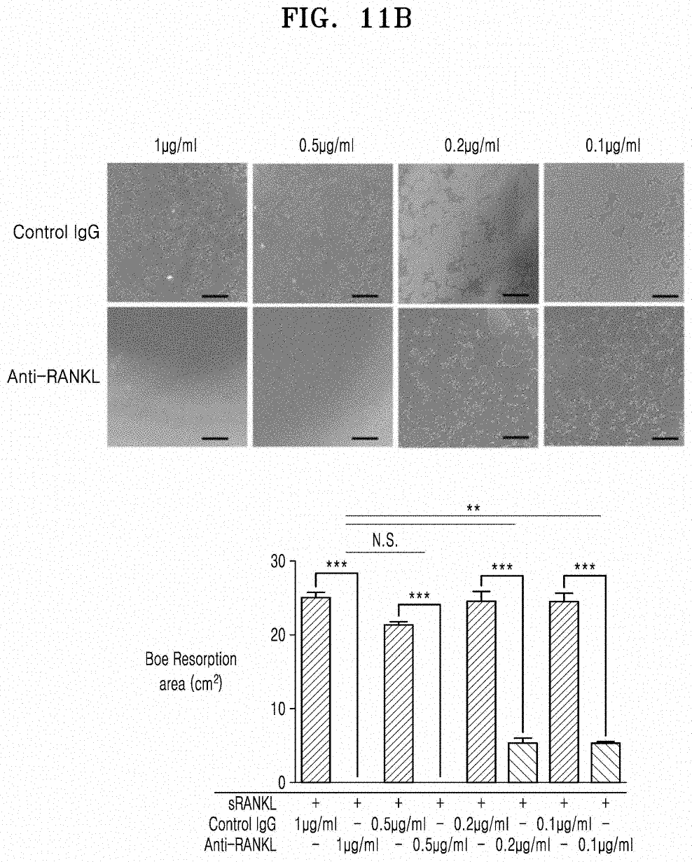

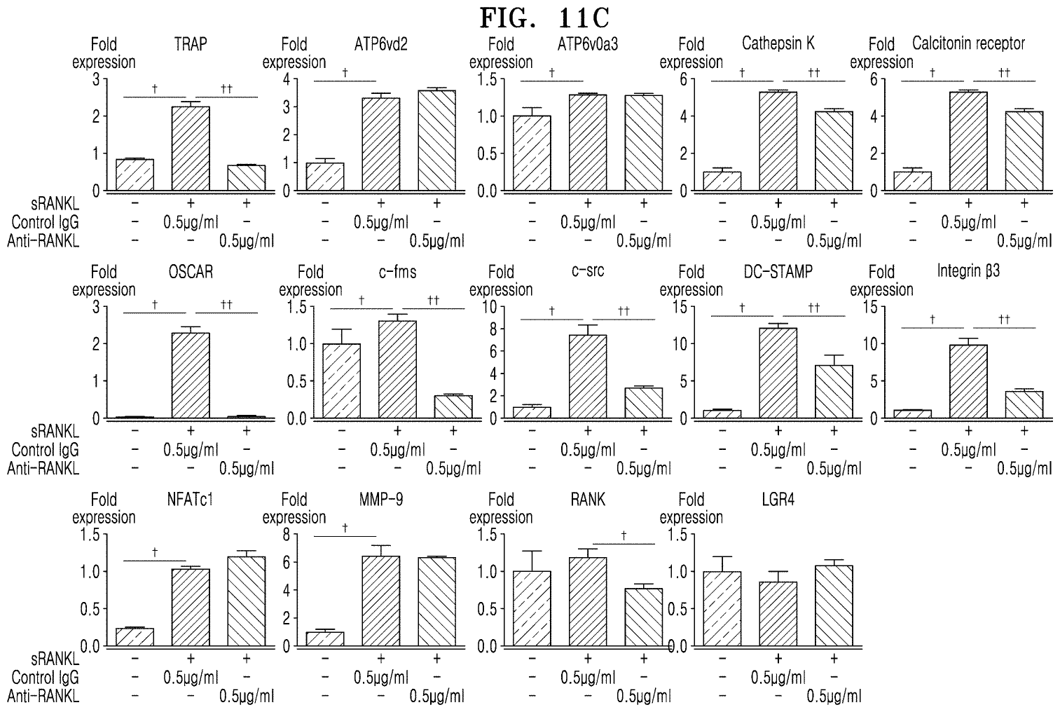

To investigate the effects of purified anti-RANKL on the inhibition of osteoclastogenesis, primary BMMs were treated with purified anti-RANKL from mRANKL-MT3-immunized mice in the presence of RANKL and M-CSF. While commercial control IgG had no effect on osteoclastogenesis, TRAP-positive multinucleated cells and bone resolving area were significantly reduced by anti-RANKLtreated BMMs, even at 0.1 μg/mL (FIG. 11A, 11B). In addition, anti-RANKL from mRANKL-MT3-immunized mice significantly decreased TRAP and OSCAR mRNA expression (FIG. 11C). However, anti-RANKL did not decrease NFATc1 levels.

### REFERENCE EXAMPLE 8

### Co-Immunoprecipitation Assay

BMM cells were incubated with 500 ng/mL mRANKLWT or mRANKL-MT3 at 37.0 for 45 min. Then, the cells were lysed in lysis buffer (20 mM Tris-HCl pH 7.6-8.0, 100 mM NaCl, 300 mM sucrose, and 3 mM MgCl₂ [buffer A] and 20 mM Tris pH 8.0, 100 mM NaCl, and 2 mM EDTA [buffer B]). Whole-cell lysates were obtained by centrifugation and incubated with antibodies specific for RANK (Cell Signaling Technology, #14373S) and LGR4 (MyBioSource, #MBS468030) (dilution 1:100) and protein A Sepharose beads (Amersham Biosciences) for 2 h at room temperature. The immune complexes were washed three times using Tris-buffered saline-Tween buffer (TBST; 2.42 g/L, Tris-HCl; 8 g/L, 0.1% Tween 20, pH 7.6) and examined by western blotting.

### EXAMPLE 7

### Comparative Inhibitory Effects of mRANKL Variants on Wild Type RANKL-Induced Osteoclastogenesis

LGR4 is part of the LGR family, which includes another two members, follicle-stimulating hormone receptor and thyroidstimulating hormone receptor, which regulate osteoclast differentiation and activity (E J Petrie, et al. Front Endocrinol (Lausanne). 2015; 6:137.). We also investigated whether LGR4 is another RANKL receptor. Specifically, we investigated whether mtRANKL does not bind and activate RANK, but instead activates LGR4, which acts as a mild inhibitor of osteoclastogenesis.

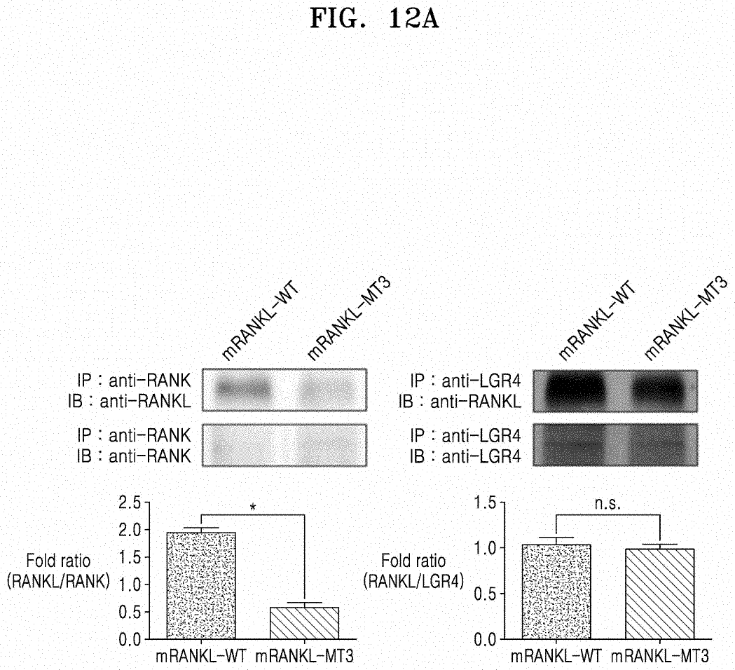

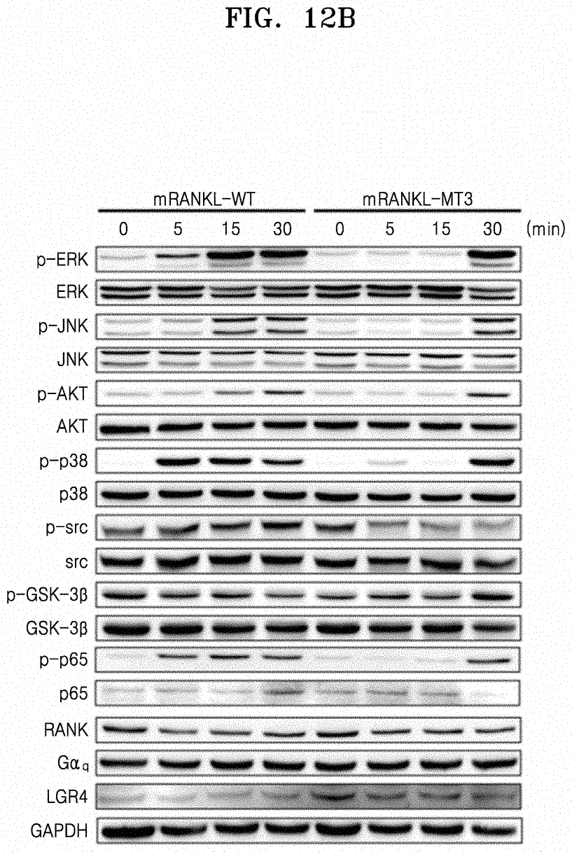

To investigate the interaction of mRANKL variants and RANK, the co-immunoprecipitation (Co-IP) assays were carried out using mRANKL-WT/mRANKL-MT3 and RANK/LGR4 (FIG. 12A). In addition, wild-type RANKL induced MAPK, AKT, NF-κB p65, and GSK-3 phosphorylation, which is involved in LGR4 signaling (FIG. 12B). Mutant mRANKL-MT3 also induced GSK-3 phosphorylation, but caused lower MAPK, AKT, and NF-κB p65 phosphorylation levels. Furthermore, src phosphorylation, which is involved in RANK signaling, was significantly lower in mRANKL-MT3-induced BMMs than in mRANKL-WTinduced BMMs. These data suggest that mRANKL-MT3 employs signaling transfer from LGR4 via src phosphorylation and without RANK signaling, in contrast to mRANKL-WT.

### EXAMPLE 8

### Effect of Anti-RANKL on RANK and LGR4 Signaling

We verified that anti-RANKL suppressed RANK and LGR4 signaling in a time-dependent manner (FIG. 13A). Treatment with sRANKL induced MAPK, AKT, src, and GSK-3β phosphorylation. However, anti-RANKL treatment significantly inhibited MAPK, AKT, src, and GSK-3β phosphorylation. In addition, NFATc1 nuclear translocation was not detected by Western blot analysis or confocal microscopy in anti-RANKL-treated BMMs in the presence of sRANKL compared with control IgG (FIG. 13B, 13C). Cytosolic calcium influx was also decreased by antiRANKL treatment in the presence of sRANKL (FIG. 13D). Finally, we demonstrated that purified anti-RANKL from mRANKL-MT3-immunized mouse sera blocked RANKL-RANK and LGR4 signaling.

1. A mutant having one or more substitutions selected from the group consisting of substitution of arginine (Arg) for lysine (Lys) at position 180, substitution of any one of isoleucine (Ile), leucine (Leu), and asparagine (Asn) for aspartic acid (Asp) at position 189, substitution of lysine (Lys) for arginine (Arg) at position 190, substitution of phenylalanine (Phe) or tyrosine (Try) for histidine (His) at position 223, and substitution of phenylalanine (Phe) or tyrosine (Try) for histidine (His) at position 224 at the N-terminus of receptor activator of nuclear factor-kappa B (NF-κB) ligand (RANKL) protein comprising an amino acid sequence of SEQ ID NO: 1, or
a mutant having one or more substitutions selected from the group consisting of substitution of arginine (Arg) for lysine (Lys) at position 181, substitution of any one of isoleucine (Ile), leucine (Leu), and asparagine (Asn) for aspartic acid (Asp) at position 190, substitution of lysine (Lys) for arginine (Arg) at position 191, substitution of phenylalanine (Phe) or tyrosine (Try) for histidine (His) at position 224, and substitution of phenylalanine (Phe) or tyrosine (Try) for histidine (His) at position 225 at the N-terminus of RANKL protein comprising an amino acid sequence of SEQ ID NO: 2.

2. The mutant of claim 1, wherein the mutant is administered to a subject to produce an antibody.

3. An antibody produced by the mutant of claim 1.

4. A nucleic acid molecule encoding the mutant of claim 1.

5. The nucleic acid molecule of claim 4, wherein the nucleic acid molecule has a nucleotide sequence of SEQ ID NO: 3.

6. The nucleic acid molecule of claim 4, wherein the nucleic acid molecule has a nucleotide sequence of SEQ ID NO: 4.

7. A vector comprising the nucleic acid molecule of claim 4.

8. A vector comprising the nucleic acid molecule of claim 5.

9. A vector comprising the nucleic acid molecule of claim 6.

10. A host cell comprising the vector of claim 6.

11. A pharmaceutical composition for preventing or treating a metabolic bone disease, the pharmaceutical composition comprising, as active ingredient, the mutant of claim 1.

12. A pharmaceutical composition for preventing or treating a metabolic bone disease, the pharmaceutical composition comprising, as active ingredient, the antibody of claim 3.

13. The pharmaceutical composition of claim 8, wherein the metabolic bone disease is one or more selected from the group consisting of osteoporosis, osteodystrophy, and bone fracture of humans.

14. The pharmaceutical composition of claim 9, wherein the osteoporosis is caused by inducing differentiation of osteoclasts due to binding of nuclear factor kappa-B ligand (RANKL) to receptor activator of nuclear factor-kappa B (RANK).

15. The pharmaceutical composition of claim 8, wherein the mutant is administered to a subject to produce an antibody.

16. The pharmaceutical composition of claim 8, wherein the mutant is an antagonist of RANK receptor in a subject.

17. The pharmaceutical composition of claim 8, wherein the mutant is administered to a subject not to induce differentiation of osteoclasts.
